(19) United States
(12) Patent Application Publication
(10) Pub. No.: US 2021/0236995 A1
(43) Pub. Date: Aug. 5, 2021

(54) A SEMIPERMEABLE ARRANGEMENT

(71) Applicant: Ernest Wai Yin Lau, Belfast (GB)

(72) Inventor: Ernest Wai Yin Lau, Belfast (GB)

(21) Appl. No.: 17/052,629

(22) PCT Filed: May 3, 2019
(86) PCT No.: PCT/EP2019/061465

§ 371 Date: Nov. 3, 2020

(30) Foreign Application Priority Data
May 3, 2018 (GB) 1807322.1
Aug. 7, 2018 (GB) 1812846.2

(51) Int. Cl. B01D 65/08; B01D 71/36; B01D 69/02; B01D 69/14

(52) U.S. Cl. CPC B01D 65/08; B01D 71/36; B01D 69/02; A61F 2002/018; B01D 2325/48; B01D 2325/08; B01D 69/141

(57) ABSTRACT

A semipermeable arrangement for use in clinical, agricultural, industrial and/or environmental settings. The semipermeable arrangement has a structural arrangement formed from a material such as ePTFE that has an affinity to a lubricating fluid such as perfluorocarbon. The structural arrangement may be infused with a lubricating fluid such that the semipermeable arrangement resists fouling. The semipermeable arrangement is further arranged with barriers to prevent or limit the movement of the lubricating fluid through at least part of the structural arrangement. The semipermeable arrangement further has passageways that are free from the presence of, and/or cannot be infused with, lubricating fluid. The passageways permit the movement of fluids such as air, water and dissolved substances through the structural arrangement. The semipermeable arrangement is thereby both self-cleaning and porous and has a wide range of uses.

The present invention relates to a semipermeable arrangement for use in clinical, agricultural, industrial and/or environmental settings.

A semipermeable arrangement, such as a porous membrane, restricts the passage of materials across it to only certain selected substances. Typically, selected substances include water and dissolved substances, and solid particles and microbes are prevented from crossing the porous membrane. However, the excluded particles and microbes can settle on the inlet side of the porous membrane and the microbes can colonise the surface, clogging up the membrane's pores (“fouling”). When sufficient materials build up and/or microbes proliferate on the membrane surface, the membrane can no longer function effectively. Where the porous membrane is used in a clinical setting, for example being applied or implanted on a recipient, it may become a source of microbial infection and needs to be removed and replaced. Removal and replacement of the porous membrane may be technically difficult, costly and even dangerous if it is encased in the recipient's tissue. Specific technologies have been developed to protect surfaces from fouling (generally by making them less easy to stick to or self-cleaning, and/or by applying antimicrobial substances to the surface), but they may not be compatible with the functional requirements of a porous membrane if the selected substances will also be prevented from passing through it. In addition, some self-cleaning materials such as polytetrafluoroethylene (PTFE) are inflexible, which can render them unsuitable for some applications, and it is generally desirable to avoid the use of antimicrobials where possible as this can encourage the emergence of antimicrobial resistance. There is accordingly a requirement for a porous self-cleaning membrane that is flexible and resists fouling with restricted use of antimicrobial substances.

A further problem potentially addressable by porous membranes resides in the area of drug-eluting materials, such as drug-eluting stents. Such materials are manufactured having a platform with a polymer coating that is bound to the drug. After insertion of the material in a recipient the drug is slowly released from the polymer over time. The manufacturing process of such materials is timely and often costly. If the drug does not bind correctly to the polymer, or is degraded during the polymerization process, the polymer must be redesigned, or the drug cannot be used. There is a requirement, therefore, for an alternative solution to drug eluting materials with an improved manufacturing process.

A further area that could benefit from improved semipermeable membranes regards Medical and Veterinary Implantable Electronic Devices (MAVIEDs) such as cardiac pacemakers, implantable cardioverter-defibrelators (ICDs), internal loop recorders, spinal cord stimulators, vagal nerve stimulators and deep brain stimulators that are inserted into the human or animal body for medical and veterinary purposes. MAVIEDs typically comprise a “pulse generator” (the battery-electronic circuitry unit) placed in an anatomically easily accessible pocket (e.g. under the skin, in subcutaneous tissues or under skeletal muscles) and connected by one or more “leads” (electric cords containing insulated conductors and electrodes exposed on the surface) to anatomically remote sites within the body. Over time, the pulse generator and leads in the pocket become encased within a fibrous “capsule”, which can make pulse generator and lead replacement or removal difficult and can cause infection or component failure.

A further problem is that MAVIED leads are prone to failure following sufficient wear and/or abrasion. Numerically, outside-in abrasion, i.e. where the abrasive force is resulting from forces acting on the surface of the recipient's skin, within the surgical pocket that houses redundant lead lengths and the pulse generator is the most important cause of lead insulation failure. Traditionally, the redundant lead lengths are inserted under the pulse generator inside the surgical pocket. While this “lead-bottom” intra-pocket arrangement protects the leads from accidental damage by sharp instruments when the surgical pocket is re-opened for MAVIED revision, the leads are sandwiched between the pulse generator and the recipient's bone skeleton and hence more prone to outside-in abrasion. In theory, the redundant lead lengths can be inserted on top of the pulse generator inside the surgical pocket. This “lead-top” intra-pocket arrangement will leave the leads sandwiched between the soft skin and subcutaneous tissues, which should protect the leads from outside-in abrasion. However, the redundant lead lengths may not only be damaged by sharp instruments when the surgical pocket is re-opened, but also become embedded into the roof of the surgical pocket by fibrous tissues and very difficult to mobilize at the time of MAVIED revision. For these reasons, the alternative “lead-top” intra-pocket arrangement is rarely adopted in clinical practice. Covering the MAVIED and the lead in the “lead-top” intra-pocket arrangement is one potential solution. However, the material used to cover the MAVIED should ideally be self-cleaning to prevent adhesion of microbes or build-up of fibrous tissue, as well as being permeable to water and dissolved substances such that the MAVIED can remain electrically connected to the recipient's body and electrodes on the leads. No such material has existed previously.

It is an object of the invention to mitigate or obviate the problems associated with semipermeable arrangements.

It is a further object of the invention to mitigate or obviate the problems associated with fouling of semipermeable arrangements.

It is a further object of the invention to mitigate or obviate the problems associated with use of semipermeable arrangements for the purpose of filtration.

It is a further object of the invention to mitigate or obviate the problems associated with use of semipermeable arrangements for use in clinical settings.

It is a further object of the invention to mitigate or obviate the problems associated with materials adapted for controlled release of chemical substances.

It is a further object of the invention to mitigate or obviate the problems associated with wear and damage to MAVIEDs and associated leads, in particular, from outside-in abrasion.

It is a further object of the invention to mitigate or obviate the problems associated with fibrous tissue formation and tissue ingrowth on MAVIEDs and associated leads.

It is a further object of the invention to mitigate or obviate the problems associated with biofilm formation on MAVIEDs and associated leads.

It is a further object of the invention to mitigate or obviate the problems associated with biofilm formation on MAVIEDs and associated leads without the use of chemical antimicrobial agents.

It is a further object of the invention to mitigate or obviate the problems with lead-top intra-pocket arrangements.

It is a further object of the invention to mitigate obviate the problems associated with explantation of medical devices.

According to a first aspect of the invention there is provided a semipermeable arrangement for use in clinical, agricultural, industrial and/or environmental settings comprising a structural means and at least one porous member, the at least one porous member being located on, within and/or supported by the structural means.

Ideally, at least part of the structural means is impermeable to water and dissolved substances.

Advantageously, passage of water and dissolved substances through the membrane occurs via the porous member(s). Altering the properties of the porous member(s) can alter the flow-rate and filtration capabilities of the semipermeable arrangement.

In one embodiment, the structural means is arrangeable as a layer, most preferably, comprising a single layer.

In another embodiment, the structural means comprises a plurality of layers.

Ideally, the plurality of layers are interconnected and/or are arranged adjacent to each other.

Preferably, the plurality of layers are arrangeable as a three-dimensional geometric shape such as a sphere, cube, cuboid or polyhedron.

Preferably, the structural means comprises a means for retaining and/or supporting the at least one porous member. By retaining we mean holding in place, either by directly abutting and retaining or by acting on a secondary structure which abuts and retains the porous member.

Preferably, the structural means defines a structural plate, structural box, structural frame, clamp and/or other suitable geometric formation or combination thereof that provides structure and is capable of supporting at least one porous member.

Ideally, the porous member retaining means comprises structural rods.

Preferably, the structural rods comprising a diameter/width between 0.1 and 10 mm.

In one embodiment, the structural rods comprising a diameter/width between 0.5 and 5 mm.

Ideally, the structural rods comprising a diameter/width of around 1 mm.

In another embodiment, the structural rods comprising a diameter/width between 0.1 and 1 mm.

Ideally, the structural rods comprising a diameter/width of around 0.4 mm.

Preferably, the structural rods comprising an average diameter/width of around 0.39 mm.

Ideally, the structural rods are spaced apart.

Ideally, the structural means, most preferably, the structural rods comprise a polymeric substance.

Ideally, the structural means, most preferably, the structural rods comprise a fluoropolymer.

Ideally, the structural means, most preferably, the structural rods comprise polytetraofluoroethylene (PTFE), most preferably, expanded PTFE (ePTFE).

Preferably, the structural rods are polymeric rods.

Preferably, the structural rods are fluoropolymer rods.

Preferably, the structural rods are formed from ePTFE.

Advantageously, ePTFE is flexible relative to PTFE and therefore the structural rods and the structural frame are flexible and easy to handle.

Ideally, the structural rods are ePTFE rods.

Ideally, the structural rods have a circular or oval cross-section.

In one embodiment, the structural rods are arranged as a mesh.

Ideally, the structural frame comprises two groups of structural rods.

Ideally, the structural frame comprises a first group of structural rods and a second group of structural rods.

Preferably, the first group of structural rods comprises a plurality of structural rods in a spaced apart, parallel arrangement.

Ideally, the second group of structural rods comprises a plurality of structural rods in a spaced apart, parallel arrangement.

Preferably, the first group of structural rods are arranged perpendicular to the second group of structural rods.

Preferably, the structural rods are reticulated.

Ideally, the structural rods are reticulated, wherein one group of spaced apart structural rods is overlaying and/or is intertwined with another group of spaced apart structural rods.

Preferably, the spacing between the structural rods in the first and/or second group of structural rods is between 0.1 and 10 mm.

Ideally, the spacing between the structural rods in the first and/or second group of structural rods is between 0.5 and 5 mm.

Preferably, the spacing between the structural rods in the first and/or second group of structural rods is between 2 and 4 mm.

Ideally, the spacing between the structural rods in the first and/or second group of structural rods is around 3 mm.

Ideally, the spacing between the structural rods in the first and/or second group of structural rods is between 0.5 and 5 mm thereby defining a mesh network having a plurality of openings, each opening having an area of between 0.25 and 25 mm².

Preferably, the spacing between the structural rods in the first and/or second group of structural rods being between 2 and 4 mm thereby defining a mesh network having a plurality of openings, each opening having an area of between 4 and 16 mm².

Preferably, the spacing between the structural rods in the first and/or second group of structural rods being around 3 mm thereby defining a mesh network having a plurality of openings, each opening having an area of around 9 mm².

In one embodiment, a plurality of mesh networks are arranged to create layers of mesh networks.

Ideally, the plurality of mesh networks are arranged as a lattice structure.

Ideally, the at least one porous member is sized such that it can be located in an opening of the mesh network.

Ideally, when the at least one porous member is located in an opening of the mesh network the mesh network abuts the porous member.

Preferably, the abutment between the mesh network and the porous member retains the porous member in the mesh network.

In one embodiment, the at least one porous member is retained in the mesh network via an interference fit.

Preferably, the at least one porous member is formed from a biocompatible and/or biostable substance.

Advantageously, the semipermeable arrangement is suitable for clinical use.

Preferably, the at least one porous member comprises at least one polymeric substance, most preferably, at least one thermoplastic polymeric substance.

Preferably, the at least one porous member is formed from sintering of loosely compacted powders.

Ideally, the loosely compacted powered are thermoplastic polymers.

Advantageously, this process can be modified to adjust the properties of the semipermeable arrangement. For example, the particle size will affect the rate of movement of fluid through the porous member.

Preferably, the at least one porous member comprises a plurality of particles adhered and/or sintered together.

Ideally, the plurality of particles are adhered and/or sintered together with gaps therebetween defining a pore size.

Advantageously, this enhances permeability of the porous member.

Ideally, the pore size can be modified depending on manufacturing conditions and particle size.

Preferably, the pore size is less than or equal to 250 μm.

Preferably, the pore size is less than or equal to 125 μm.

Preferably, the pore size is less than or equal to 75 μm.

Preferably, the pore size is less than or equal to 50 μm.

Advantageously, matter having a dimension greater than 50 μm cannot filter through the at least one porous member. It will be understood that a small powder particle size will result in smaller spacings between the particles and a smaller average overall pore size. This reduces the average cross-sectional area of the pore size and will reduce the flow rate through the porous member and therefore the semipermeable arrangement. The manufacturing process of the porous member(s) can be adjusted to produce the desired properties in the semipermeable arrangement.

Preferably, the at least one porous member is a three-dimensional geometric shape.

In one embodiment, at least one porous member is spherical, roughly spherical, or a flattened sphere, spherical cap, hemisphere, ovoid, cube or cuboid in shape.

Alternatively, at least one porous member is disc-shaped.

In one embodiment, the structural means defines a plane.

In this embodiment, the porous member is arranged relative to the structural means such that the shortest dimension of the porous member is substantially perpendicular to the plane of the structural means.

Ideally, the porous member has a diameter or width of about 1.5 mm.

Preferably, the porous member has a thickness greater than that of the structural means.

Advantageously, generally any three-dimensional geometric shape with a thickness greater than the structural means can be used provided it is capable of interacting with the structural means.

Ideally, the porous member has a thickness of equal to or greater than 0.25 mm.

Preferably, the porous member has a thickness equal to or greater than 0.4 mm.

Ideally, the porous member has a thickness equal to or greater than 0.75 mm.

In one embodiment, the porous member has a thickness of about 1 mm.

It will be understood that the semipermeable arrangement may comprise a range of shapes and sizes of porous members.

In one embodiment, the at least one porous member is sized between 0.1 and 10 mm, most preferably, the at least one porous member is spherical, roughly spherical, a flattened sphere, spherical cap, hemisphere, ovoid or disc-shaped having a diameter/width between 0.1 and 10 mm.

In one embodiment, the at least one porous member is sized between 0.5 and 8 mm, most preferably, the at least one porous member is spherical, roughly spherical, a flattened sphere, spherical cap, hemisphere, ovoid or disc-shaped having a diameter/width between 0.5 and 8 mm.

Ideally, the at least one porous member is sized between 1 and 5 mm, most preferably, the at least one porous member is spherical, roughly spherical, a flattened sphere, spherical cap, hemisphere, ovoid or disc-shaped having a diameter/width between 1 and 5 mm.

Ideally, the at least one porous member is sized around 3 mm, most preferably, the at least one porous member is spherical, roughly spherical, a flattened sphere, spherical cap, hemisphere, ovoid or disc-shaped having a diameter/width of about 3 mm.

Ideally, the at least one porous member extends out of the plane of the structural means, either above and/or below the plane.

Preferably, the at least one porous member has a raised profile relative to the plane of the structural means.

Ideally, the at least one porous member extends out of the plane of the structural means by at least 0.05 mm.

In one embodiment, the at least one porous member extends out of the plane of the structural means by at least 0.5 mm.

Ideally, the at least one porous member extends out of the plane of the structural means by about 1 mm.

Advantageously, in one embodiment, the at least one porous member has a diameter/width of 3 mm and the structural rods have a diameter/width of around 1 mm, so the at least one porous member extends out of the plane of the structural means by around 1 mm at either side of the structural means. This increases the surface area of the semipermeable arrangement providing a large filtration surface. Additionally, the domed shape of the porous members within the structural means prevent matter from settling as matter will be predisposed to rolling off the surface of the dome shape.

In another embodiment, the at least one porous member extends out of the plane of the structural means by about 0.3 mm.

Advantageously, in this embodiment the porous member is almost flush with the surface of the structural means. The thickness of the semipermeable arrangement is close to being equal to the thickness of the structural means and this creates a more aesthetically pleasing product. In addition, where the semipermeable arrangement is to be implanted inside the human or animal body, the extension of the porous members beyond the plane of the structural means is minimized and the semipermeable arrangement does not excessively protrude from the site of implantation. This can result in greater comfort for the recipient.

In one embodiment, the semipermeable arrangement comprises one or more chemical substances.

Ideally, the semipermeable arrangement comprises soluble particles of one or more chemical substances.

Preferably, the at least one porous member comprises soluble particles of one or more chemical substances.

In one embodiment, the soluble particles are crystalized particles.

Ideally, the soluble particles are disposed on the exterior and/or interior/pore surfaces of the porous member.

Preferably, the one or more chemical substances comprises at least one pharmacological and/or antimicrobial agent.

In one embodiment, the one or more chemical substances comprises a carrier substance.

Advantageously, the semipermeable arrangement can be arranged such that it is drug eluting. In comparison to preparation of other drug-eluting materials, the drug is not required to be bound to a polymer and comparatively the manufacturing process is remarkably quick and simple. Additionally, the drug only need not to react with a polymer. Simply, the porous members can be exposed or submerged to a solution containing the drug and allowed to dry. The solvent evaporates and the solute drug crystallizes within the physical interstices of the polymer. Where the semipermeable arrangement is used in a clinical setting, the soluble particles will gradually dissolve and release the one or more chemical substances. The addition of a carrier can be used to control the rate of release. This feature can be used to further enhance the antimicrobial properties of the semipermeable arrangement or to provide treatment to a recipient, and provides an additional or alternative, cost-effective solution to drug administration and drug-eluting materials. Furthermore, the pore size and void volume of the porous members are adjustable by altering the manufacturing process. This can be used to increase or decrease the quantity of additional chemical substances, such as antibiotics, that can be contained within the porous members and/or the rate of elution out of the porous members into the surrounding medium.

Ideally, as the soluble particles dissolve the pore size increases.

Advantageously, where the semipermeable arrangement is being used in a clinical setting and after it has been inserted on or into a recipient, the permeability of the semipermeable arrangement increases over time as the soluble particles are dissolved.

Ideally, the semipermeable arrangement comprises a plurality of porous members.

Ideally, the plurality of porous members are located interspersed on or within the structural means.

Preferably, the plurality of porous members are located in a spaced apart relationship on or within the structural means.

Preferably, the plurality of porous members are located in a spaced apart relationship on or within the mesh network of the structural frame.

Ideally, the plurality of porous members are arranged in a pattern within and/or on the structural means.

Ideally, the plurality of porous members are arranged in or on the structural frame adjacent to an opening in the structural frame, the opening being vacant from any porous member.

Preferably, the plurality of porous members are arranged in rows within the structural means.

Preferably, the plurality of porous members are arranged in rows within the structural frame such that every other opening in each row of the mesh network of the structural frame contains a porous member.

Preferably, the plurality of porous members are arranged within the structural frame in a chequered pattern such that every other opening in the row of the mesh network of the structural frame contains a porous member and that a row above or below this row contains a similar pattern but is offset relative to the row such that the plurality of porous members are arranged in a diagonal relationship within the mesh network but not laterally or longitudinally adjacent to one another.

Ideally, the semipermeable arrangement comprises a filter means.

In one embodiment, the filter means is provided by the structural means.

Ideally, the filter means can filter particles of matter from a fluid.

Preferably, the filter means can exclude particles or microbes less than 1.0 μm in size from entering the at least one porous member.

Ideally, the filter means comprises a filter web having spacings, the spacings being equal to or less than 2.0 μm.

Ideally, the filter means comprises a filter web having spacings, the spacings being equal to or less than 1.0 μm.

Preferably, the filter means comprises a filter web having spacings, the spacings being equal to or less than 0.5 μm.

Advantageously, a filter web spacing of equal to or less than 0.5 μm provides a satisfactory trade-off between permeability and filtration capabilities, wherein bacteria including Staphylococcus aureus and other pertinent pathogens are prevented from passing through the web, but wherein an acceptable flow-rate of fluid through the web is maintained. The thinness of the filter web (e.g. 0.1-0.3 μm) permits particles with diameter <0.5 μm to pass through.

In one embodiment, the filter means comprises a filter web having spacings, the spacings being equal to or less than 0.2 μm.

Advantageously, the filter means is permeable to gas, water and dissolved substances but not large particles and bacteria.

Preferably, the filter means, most preferably the filter web, is arranged on a surface of the structural means and/or the at least one porous member.

In one embodiment, the filter means, most preferably the filter web, is arranged on a plurality of surfaces of the structural means and/or the least one porous member.

Preferably, the filter web comprises at least one polymeric substance.

Ideally, the filter web is formed by electrospinning.

Preferably, the filter web is formed from electrospinning of polymers.

Preferably, the filter web comprises a plurality of filter web fibres.

By fibres we mean any threadlike form and including fibrils or filaments.

Preferably, the filter web comprises a plurality of filter web fibres irregularly arranged.

Ideally, the filter web comprises a plurality of filter web fibres arranged overlapping one another and having spacings therebetween.

Ideally, the filter web comprises a plurality of filter web fibres arranged overlapping one another and having spacings therebetween, the spacings being equal to or less than 2.0 μm.

Ideally, the filter web comprises a plurality of filter web fibres arranged overlapping one another and having spacings therebetween, the spacings being equal to or less than 1.0 μm.

Ideally, the filter web comprises a plurality of filter web fibres arranged overlapping one another and having spacings therebetween, the spacings being equal to or less than 0.5 μm.

Ideally, the filter web comprises a plurality of filter web fibres arranged overlapping one another and having spacings therebetween, the spacings being equal to or less than 0.2 μm.

Preferably, the filter means is biocompatible and/or biostable.

Advantageously, the filter means is suitable for clinical use.

Preferably, the filter web fibres are polymer fibres.

Ideally, during electrospinning of the filter web, the average diameter/width of filter web fibres can be controlled by altering the syringe arrangement.

Ideally, the filter means is formed at least partially from a polymeric substance that does not contain fluorine atoms.

Advantageously, the filter means does not have an affinity to perfluorocarbons.

Ideally, the filter means is formed at least partially from polyurethane.

Preferably, the filter means is formed at least partially from electrospun polyurethane.

Preferably, the size of the spacing between the filter web fibres can be controlled via the manufacturing process and through stretching after manufacture.

Preferably, the filter web fibres have an average diameter/width of between 0.01 and 100 μm.

Preferably, the filter web fibres have an average diameter/width of between 0.01 and 10 μm.

Ideally, the filter web fibres have an average diameter/width of about 0.1 μm.

In one embodiment, the filter means is formed, placed and/or stretched over the structural means and/or the at least one porous member.

In one embodiment, wherein the structural means is planar, the filter means is located on at least both planar surfaces of the structural means wherein the structural means is sandwiched by the filter means.

Alternatively, the filter means may be located within/between the structural means such that filter means is sandwiched by all or part of the structural means.

In one embodiment, the spacing between the filter web fibres is wider at or around the at least one porous member than the spacing between the filter web fibres at or around the structural means.

Ideally, the filter web comprises a plurality of filter web fibres arranged overlapping one another and having spacings therebetween, the spacing being equal to or less than 2.0 μm.

Ideally, the filter web comprises a plurality of filter web fibres arranged overlapping one another and having spacings therebetween, the spacing being equal to or less than 1.0 μm.

Ideally, the filter web comprises a plurality of filter web fibres arranged overlapping one another and having spacings therebetween, the spacing being equal to or less than 0.5 μm.

Ideally, the filter web comprises a plurality of filter web fibres arranged overlapping one another and having spacings therebetween, the spacing being equal to or less than 0.2 μm.

Preferably, the filter means has a melting point less than or equal to 330° C.

Ideally, the filter means has a melting point less than or equal to 200° C.

Ideally, the filter means has a melting point less than or equal to 150° C.

Ideally, the filter means has a melting point less than or equal to 100° C.

Advantageously, parts of the filter means can be melted to adhere it to the structural means and/or other component parts of the semipermeable arrangement, or for melting one portion of the filter means to another portion of the filter means.

Preferably, the semipermeable arrangement comprises a self-cleaning means.

By self-cleaning we mean repels substances that result in fouling. Advantageously, the self-cleaning means reduces the rate of fouling of the semipermeable arrangement or obviates it entirely.

In one embodiment, the self-cleaning means is provided by the structural means.

Ideally, the self-cleaning means comprises a self-cleaning web.

Ideally, the self-cleaning means, most preferably, the self-cleaning web is arranged on a surface of the filter means and/or the at least one porous member.

Ideally, the self-cleaning means, most preferably the self-cleaning web, is arranged on a plurality of surfaces of the filter means and/or the at least one porous member.

In one embodiment, the self-cleaning means is arranged on a surface of some of the porous members but not all the porous members.

In another embodiment, the self-cleaning means, most preferably, the self-cleaning web is arranged on a surface of the structural means.

Preferably, the self-cleaning means, most preferably the self-cleaning web, is arranged on a plurality of surfaces of the structural means.

In one embodiment, wherein the structural means is planar, the self-cleaning means is located on at least both planar surfaces of the structural means wherein the structural means is sandwiched by the self-cleaning means.

Alternatively, the self-cleaning means may be located within/between the structural means such that the self-cleaning means is sandwiched by all or part of the structural means.

It will be understood that in this embodiment portions of the structural means that are not covered by the self-cleaning means may be subject to fouling, and so the semipermeable arrangement may be only partially self-cleaning.

In another embodiment, the filter means, most preferably the filter web, is arranged on a surface of the self-cleaning means and/or the at least one porous member.

Preferably, the filter means, most preferably the filter web, is arranged on a plurality of surfaces of the self-cleaning means and/or the at least one porous member.

Ideally, at least part of the filter means is adhered to at least part of the self-cleaning means and/or the structural means.

Ideally, at least part of the filter means is bound to at least part of the self-cleaning means and/or the structural means, most preferably by the components being pressed and/or melted together.

Advantageously, the filter means provides additional structure to the semipermeable arrangement by holding together the self-cleaning means and the structural means.

Alternatively, an adhesive, melted substance and/or mechanical clamp in addition to or instead of the filter means can be provided to hold parts of the self-cleaning means together and/or to hold the self-cleaning means and the structural means together.

In one embodiment, the semipermeable arrangement comprises a means for retaining the self-cleaning means, the structural means and/or the filter means together.

Ideally, the means for retaining the self-cleaning means, the structural means and/or the filter means together comprises a clamp, a mechanical fixing means, adhesives or other such means fitted to the self-cleaning means, the structural means and/or the filter means.

Preferably, the self-cleaning means, most preferably the self-cleaning web, comprises at least one polymeric substance.

Ideally, the self-cleaning web is formed by electrospinning.

Preferably, the self-cleaning web is formed by electrospinning of polymers.

Preferably, the self-cleaning web comprises a plurality of self-cleaning web fibres.

Preferably, the self-cleaning web comprises a plurality of self-cleaning web fibres irregularly arranged.

Ideally, the self-cleaning web comprises a plurality of self-cleaning web fibres arranged overlapping one another and having spacings therebetween.

Preferably, the spacings being formed to enable capillary action of liquid, most preferably, of perfluorocarbon liquids.

Ideally, the self-cleaning web comprises a plurality of self-cleaning web fibres arranged overlapping one another and having spacings therebetween, the spacing being equal to or less than 2.0 μm.

Ideally, the self-cleaning web comprises a plurality of self-cleaning web fibres arranged overlapping one another and having spacings therebetween, the spacing being equal to or less than 1.0 μm.

Ideally, the self-cleaning web comprises a plurality of self-cleaning web fibres arranged overlapping one another and having spacings therebetween, the spacing being equal to or less than 0.5 μm.

Advantageously, in one embodiment wherein the self-cleaning web is untreated with lubricating fluid, a self-cleaning web spacing of equal to or less than 0.5 μm provides a satisfactory trade-off between permeability and filtration capabilities, wherein bacteria including Staphylococcus aureus and other pertinent pathogens are prevented from passing through the web, but wherein an acceptable flow-rate of fluid through the web is maintained. Ideally, the self-cleaning web comprises a plurality of self-cleaning web fibres arranged overlapping one another and having spacings therebetween, the spacing being equal to or less than 0.2 μm.

Preferably, the self-cleaning means is biocompatible and/or biostable.

Advantageously, the self-cleaning means is suitable for clinical use.

Preferably, the self-cleaning web fibres are polymer fibres.

Ideally, the average diameter/width of the self-cleaning web fibres can be controlled during the manufacture process.

Preferably, the self-cleaning web fibres have an average diameter/width of between 0.01 and 100 μm.

Preferably, the self-cleaning web fibres have an average diameter/width of between 0.01 and 10 μm.

Ideally, the self-cleaning web fibres have an average diameter/width of about 0.1 μm.

Ideally, the self-cleaning means is formed at least partially from a polymeric substance that contains fluorine atoms.

Advantageously, the self-cleaning means has an affinity to perfluorocarbons.

Ideally, the self-cleaning means is formed at least partially from PTFE.

Preferably, the self-cleaning means is formed at least partially from expanded or electrospun (es) PTFE.

Advantageously, PTFE is omniphobic and repels both hydrophobic and hydrophilic substances that could result in fouling.

Preferably, the size of the spacing between the self-cleaning web fibres can be controlled via the manufacturing process and through stretching.

Ideally, the self-cleaning web is formed, placed and/or stretched over the filter means and/or the at least one porous member.

In one embodiment, the self-cleaning web is formed, placed and/or stretched over the to structural means and/or the at least one porous member.

Advantageously, the self-cleaning means prevents degradation of at least part of the filter means by isolating a part of the filter means from the surrounding environment.

In one embodiment, the semipermeable arrangement comprises at least one supplementary substance.

Ideally, the at least one supplementary substance comprises a lubricant.

Preferably, the self-cleaning means comprises at least one supplementary substance.

Ideally, the at least one supplementary substance is hydrophobic or omniphobic.

Ideally, the lubricant is hydrophobic or omniphobic.

Advantageously, the omniphobicity of the lubricant repels both hydrophobic and hydrophilic substances, thereby eliminating the possibility of fouling and preventing biofilm formation or tissue ingrowth. Further advantageously, biofilm formation can be prevented without the use of any antimicrobials, accordingly use of the semipermeable arrangement does not contribute to complications resulting from use of certain antimicrobials and/or to the emergence of antimicrobial-resistant bacteria.

Preferably, the at least one supplementary substance has an affinity for the self-cleaning web and/or the support frame.

Preferably, the at least one supplementary substance comprises perfluorocarbon liquid.

Ideally, the perfluorocarbon liquid comprises perfluoropolyether (PFPE), perfluoroperhydrophenanthrene (PFPH), perfluorodecalin (PFD) and/or other perfluorocarbon compounds.

Advantageously, perfluorocarbons are chemically relatively inert and have a chemical affinity for PTFE, ePTFE and electrospun PTFE. In an embodiment where the support frame and/or the self-cleaning web are formed from PTFE, the lubricant has a natural affinity for these components. Perfluorocarbons have been used in both medical and veterinary and consumer products. PFPH has been used for retinal tear repair in humans and in cosmetics such as lipsticks, glosses and eye shadows (e.g. Tefpoly®, The Innovation Company, Dreux, France) and PFD has been studied as a blood substitute.

Preferably, the self-cleaning web is saturated with at least one supplementary substance.

Preferably, the at least one supplementary substance is dispersed throughout the self-cleaning web by capillary action.

Ideally, the at least one supplementary substance is held throughout the self-cleaning web by capillary action.

In one embodiment, the structural means is saturated with the at least one supplementary substance.

In one embodiment, the spacing between the self-cleaning web fibres is wider at or around the at least one porous member than the spacing between the self-cleaning fibres at or around the structural means and/or filter means.

Preferably, the spacing between the self-cleaning web fibres at, around or covering the at least one porous member is too large to support capillary uptake of the at least one supplementary substance.

Advantageously, the at least one supplementary substance is not located covering the at least one porous member. This enables movement of water and dissolved substances into and through the at least one porous member.

In one embodiment, the at least one supplementary substance is present throughout the structural means in sufficient quantity such that the volume of the at least one supplementary substance is redistributed throughout the structural means when the structural means is misshapen, deformed and/or bent, so that the at least one supplementary substance infusing a part of the surface of the semipermeable arrangement is not diminished by bending.

Ideally, when the semipermeable arrangement is misshapen, deformed and/or bent the lubricant is redistributed around the structural means.

Advantageously, this ensures that lubricant is always present on the surfaces of the semipermeable arrangement and the slipperiness conferred by the lubricant is not diminished by bending or deforming.

Ideally, the structural means provides a reservoir for the lubricant such that the self-cleaning web is replenished with lubricant when required.

In another embodiment, the self-cleaning means comprises gaps sized and corresponding to the location of the at least one porous member in the structural means.

Advantageously, the at least one porous member is not covered by the self-cleaning means and this enables movement of water and dissolved substances into and through the at least one porous member. Further advantageously, where the semipermeable arrangement is used to cover a MAVIED in a clinical setting, the passage of ions across the semipermeable arrangement is possible via the porous member and the MAVIED can retain functionality despite being protected by the semipermeable arrangement. Microbes and fibrous tissue are prevented from accumulating around the MAVIED as a result of the function of the semipermeable arrangement.

Ideally, the gaps are large enough to prevent the at least one supplementary substance from bridging across the gap.

In one embodiment, the gaps provide corresponding gaps in the continuity of the at least one supplementary substance.

Ideally, the gaps are roughly equal in size to at least one porous member or smaller than at least one porous member.

Ideally, the gaps have width and/or diameter roughly equal to that of at least one porous member.

Preferably, the gaps have a width and/or diameter of between 1 and 5 mm

Ideally, the gaps have a width and/or diameter of about 3 mm.

According to a second aspect of the invention there is provided a semipermeable arrangement for use in clinical, agricultural, industrial and/or environmental settings comprising at least one porous member, the at least one porous member being capable of comprising additives.

Ideally, the additives comprising soluble particles of one or more chemical substances.

Ideally, the semipermeable arrangement comprises a plurality of porous members.

Preferably, the semipermeable arrangement comprises a plurality of porous members arranged on and/or within and/or supported by a structural means.

Ideally, the semipermeable arrangement is formed from biostable and/or biocompatible materials.

Advantageously, the semipermeable arrangement can be applied topically to a recipient or surgically inserted into a recipient to provide a controlled release of the one or more chemical substances onto or within the recipient.

According to a third aspect of the invention there is provided a method of manufacture of a semipermeable arrangement for use in clinical, agricultural, industrial and/or environmental settings, the method comprising forming a structural means and at least one porous member and arranging the at least one porous member to be supported by the structural means.

Preferably, the at least one porous member being arranged within or on the structural means.

In one embodiment, the method comprises forming the structural means as a single layer.

In another embodiment, the method comprises forming the structural means in a plurality of layers.

Ideally, the method comprises forming structural rods.

Preferably, the method comprises forming structural rods having a diameter/width between 0.1 and 10 mm.

Preferably, the method comprises forming structural rods having a diameter/width between 0.5 and 5 mm.

Preferably, the method comprises forming structural rods having a diameter/width of around 1 mm.

Ideally, the method comprises forming the structural frame, most preferably, the structural rods from fluoropolymer.

Ideally, the method comprises forming the structural frame, most preferably, the structural rods from PTFE.

Preferably, the method comprises stretching the structural rods to form stretched or expanded PTFE.

Advantageously, this improves the flexibility of the structural rods in the transverse direction but retains strength in the longitudinal direction.

Ideally, the method comprises forming the structural rods to have a circular or oval cross-section.

Ideally, the method comprises forming a plurality of structural rods.

Preferably, the method comprising forming a first group of structural rods and a second group of structural rods.

Ideally, the method comprises weaving the plurality of structural rods together to form a reticulated mesh.

Preferably, the method comprises weaving the first group of structural rods and the second group of structural rods together to form a reticulated mesh, the first group of structural rods being arranged in a parallel arrangement and the second group of structural rods being arranged in parallel arrangement perpendicular to the first group of structural rods.

Ideally, the method comprises intertwining the plurality of structural rods.

Ideally, the method comprises intertwining the first group of structural rods with the second group of structural rods.

Preferably, the method comprises arranging the spacing between the structural rods in the first and/or second group of structural rods to be between 0.1 and 10 mm.

Preferably, the method comprises arranging the spacing between the structural rods in the first and/or second group of structural rods to be between 0.5 and 5 mm.

Preferably, the method comprises arranging the spacing between the structural rods in the first and/or second group of structural rods to be between 2 and 4 mm.

Preferably, the method comprises arranging the spacing between the structural rods in the first and/or second group of structural rods to be around 3 mm.

Ideally, the method comprises arranging the spacing between the structural rods in the first and/or second group of structural rods between 0.5 and 5 mm thereby defining a mesh network having a plurality of openings with an area of between 0.25 and 25 mm².

Ideally, the method comprises arranging the spacing between the structural rods in the first and/or second group of structural rods between 2 and 4 mm thereby defining a mesh network having a plurality of openings with an area of between 4 and 16 mm².

Ideally, the method comprises arranging the spacing between the structural rods in the first and/or second group of structural rods around 3 mm thereby defining a mesh network having a plurality of openings with an area of around 9 mm².

In one embodiment, the method comprises producing a plurality of mesh networks and arranging as a lattice structure.

Ideally, the method comprises manufacturing the at least one porous member from a biocompatible and/or biostable substance.

Ideally, the method comprises manufacturing the at least one porous member from at least one polymeric substance.

Preferably, the method comprises manufacturing the at least one porous member from at least one thermoplastic polymeric substance.

Ideally, the method comprises manufacturing the at least one porous member by sintering loosely compacted powders.

Ideally, the method comprises manufacturing the at least one porous member by sintering loosely compacted powders of thermoplastic polymers.

Ideally, the method comprises manufacturing the at least one porous member by adhering or sintering together the plurality of particles with gaps therebetween defining a pore size.

Preferably, the method comprises manufacturing the at least one porous member having a pore size less than or equal to 125 μm.

Preferably, the method comprises manufacturing the at least one porous member having a pore size less than or equal to 75 μm.

Preferably, the method comprises manufacturing the at least one porous member having a pore size less than or equal to 50 μm.

Ideally, the method comprises manufacturing the at least one porous member having a three-dimensional geometric shape.

Ideally, the method comprises manufacturing the at least one porous member to be spherical, roughly spherical, or a flattened sphere, spherical cap, hemisphere, ovoid, cube, cuboid or disc-shaped in shape.

Ideally, the method comprises manufacturing the at least one porous member to be sized between 0.1 and 10 mm, most preferably, to be spherical, roughly spherical, flattened sphere, ovoid or disc-shaped and having a diameter/width between 0.1 and 10 mm.

Ideally, the method comprises manufacturing the at least one porous member to be sized between 0.5 and 8 mm, most preferably, to be spherical, roughly spherical, flattened sphere, ovoid or disc-shaped and having a diameter/width between 0.5 and 8 mm.

Ideally, the method comprises manufacturing the at least one porous member to be sized between 1 and 5 mm, most preferably, to be spherical, roughly spherical, flattened sphere, ovoid or disc-shaped and having a diameter/width between 1 and 5 mm.

Ideally, the method comprises manufacturing at least one porous member to be sized around 3 mm, most preferably, to be spherical, roughly spherical, flattened sphere, ovoid or disc-shaped and having a diameter/width of about 3 mm.

In one embodiment, the method comprises manufacturing a porous sheet and cutting and/or punching portions of the sheet, the cut- or punch-outs forming porous members.

In one embodiment, the method comprises forming or adding additives on and/or within the structural frame and/or the at least one porous member.

Ideally, the method comprises exposing the structural frame and/or the at least one porous member in a solution containing one or more chemical substances.

Ideally, the method comprises drying the structural frame and/or the at least one porous member.

Advantageously, as the solvent evaporates from within the at least one porous member, the solute crystalises containing the one or more chemical substances form.

Ideally, the method comprises arranging the at least one porous member on or within the structural frame.

Preferably, the method comprises inserting the at least one porous member within an opening of the mesh network of the structural frame.

Ideally, the method comprises inserting a plurality of porous members within separate openings of the mesh network of the structural frame.

Preferably, the method comprises inserting the at least one porous member within the mesh network of the structural frame such that the at least one porous member extends out of the plane of the structural frame by at least 0.5 mm.

Ideally, the method comprises arranging the plurality of porous members interspersed on or within the structural frame.

Ideally, the method comprises arranging the plurality of porous members in a spaced apart relationship on or within the structural frame.

Ideally, the method comprises arranging the plurality of porous members in a spaced apart relationship on or within the mesh network of the structural frame.

Ideally, the method comprises arranging the plurality of porous members in a pattern within and/or on the structural frame.

Ideally, the method comprises arranging the plurality of porous members in or on the structural frame adjacent to an opening in the structural frame, the opening being vacant from any porous member.

Ideally, the method comprises arranging the plurality of porous members in rows within the structural frame.

Ideally, the method comprises arranging the plurality of porous members in a row within the structural frame such that every other opening in the row of the mesh network of the structural frame contains a porous member.

Ideally, the method comprises arranging the plurality of porous members in rows within the structural frame such that every other opening in the row of the mesh network of the structural frame contains a porous member and that a row above or below this row contains a similar pattern but is offset relative to the row such that the plurality of porous members are arranged in a diagonal relationship within the mesh network but not laterally or longitudinally adjacent to one another.

Preferably, the method comprises manufacturing a filter means.

Ideally, the method comprises manufacturing a filter web.

Preferably, the method comprises manufacturing a filter web by electrospinning.

Preferably, the method comprises manufacturing a filter web by electrospinning polymers.

Preferably, the method comprises manufacturing a filter web by electrospinning biocompatible/biostable polymers.

Ideally, the method comprising adjusting the electrospinning process to alter the shape, size and/or configuration of the filter web.

Ideally, the method comprising adjusting the syringe arrangement of the electrospinning process to alter the shape, size and/or configuration of the filter web.

Preferably, the method comprising adjusting the needle size of the syringe arrangement to alter the shape, size and/or configuration of the filter web.

Ideally, the method comprising adjusting the collector plate shape, size or settings to alter the shape, size and/or configuration of the filter web.

Preferably, the method comprising adjusting the properties of the electrical current of the electrospinning process to alter the shape, size and/or configuration of the filter web.

Preferably, the method comprises manufacturing a filter web by electrospinning polymers to produce a plurality of filter web fibres.

Preferably, the method comprises manufacturing a filter web by electrospinning polymers to produce a plurality of filter web fibres irregularly arranged.

Ideally, the method comprises manufacturing a filter web with spacings equal to or less than 2.0 μm.

Ideally, the method comprises manufacturing a filter web with spacings equal to or less than 1.0 μm.

Ideally, the method comprises manufacturing a filter web with spacings equal to or less than 0.5 μm.

Ideally, the method comprises manufacturing a filter web with spacings equal to or less than 0.2 μm.

Ideally, the method comprising adjusting the electrospinning process to adjust the spacings of the filter web.

Ideally, the method comprising stretching the filter web to adjust the spacings of the filter web.

Preferably, the method comprising arranging the filter means, most preferably the filter web, is on a surface of the structural frame and/or the at least one porous member.

In one embodiment, the method comprising arranging the filter means, most preferably the filter web, on a plurality of surfaces of the structural frame and/or the least one porous member.

Ideally, the method comprises forming, placing and/or stretching the filter means over the structural frame and/or the at least one porous member.

Ideally, the method comprises forming, placing and/or stretching the filter means over the structural frame and/or the at least one porous member such that the spacing between the filter web fibres is wider at or around the at least one porous member than the spacing between the filter web fibres at or around the structural frame.

Preferably, the method comprises manufacturing a self-cleaning means.

Ideally, the method comprises manufacturing a self-cleaning web.

Preferably, the method comprises forming the self-cleaning means form at least one polymeric substance.

Ideally, the method comprises manufacturing the self-cleaning means, most preferably the self-cleaning web, by electrospinning a material, most preferably the material being polymeric.

Preferably, the method comprises manufacturing a self-cleaning web by electrospinning PTFE.

Ideally, the method comprises adjusting the electrospinning process to alter the shape, size and/or configuration of the self-cleaning web.

Ideally, the method comprises adjusting the syringe arrangement of the electrospinning process to alter the shape, size and/or configuration of the self-cleaning web.

Preferably, the method comprising adjusting the needle size of the syringe arrangement to alter the shape, size and/or configuration of the self-cleaning web.

Ideally, the method comprising adjusting the collector plate shape, size or settings to alter the shape, size and/or configuration of the self-cleaning web.

Preferably, the method comprising adjusting the properties of the electrical current of the electrospinning process to alter the shape, size and/or configuration of the self-cleaning web.

Ideally, the method comprises stretching the self-cleaning web to alter the shape, size and/or configuration of the self-cleaning web.

Ideally, the method comprises stretching the self-cleaning web to alter the spacings of the self-cleaning web.

Preferably, the method comprises manufacturing the self-cleaning web from a plurality of self-cleaning web fibres.

Ideally, the method comprises forming the self-cleaning web from a plurality of self-cleaning web fibres arranged overlapping one another and having spacings therebetween.

Ideally, the method comprises adjusting the spacings between the self-cleaning web fibres to enable capillary action of liquid, most preferably, of perfluorocarbon liquids.

Ideally, the method comprises arranging a plurality of self-cleaning web fibres overlapping one another and having spacings therebetween, the spacing being equal to or less than 2.0 μm.

Ideally, the method comprises arranging a plurality of self-cleaning web fibres overlapping one another and having spacings therebetween, the spacing being equal to or less than 1.0 μm.

Ideally, the method comprises arranging a plurality of self-cleaning web fibres overlapping one another and having spacings therebetween, the spacing being equal to or less than 0.5 μm.

Ideally, the method comprises arranging a plurality of self-cleaning web fibres overlapping one another and having spacings therebetween, the spacing being equal to or less than 0.2 μm.

Ideally, the method comprises arranging the self-cleaning means, most preferably, the self-cleaning web on a surface of the filter means and/or the at least one porous member.

Ideally, the method comprises arranging the self-cleaning means, most preferably the self-cleaning web, on a plurality of surfaces of the filter means and/or the at least one porous member.

In one embodiment, the method comprises arranging the self-cleaning means, most preferably, the self-cleaning web on a surface of the structural means.

Preferably, the method comprises arranging the self-cleaning means, most preferably the self-cleaning web, on a plurality of surfaces of the structural means.

In another embodiment, the method comprises arranging the filter means, most preferably the filter web, on a surface of the self-cleaning means and/or the at least one porous member.

Preferably, the method comprises arranging the filter means, most preferably the filter web, on a plurality of surfaces of the self-cleaning means and/or the at least one porous member.

Ideally, the method comprises forming, placing and/or stretching the self-cleaning web over the filter means and/or the at least porous member.

Ideally, the method comprises forming, placing and/or stretching the self-cleaning web over the at least one porous member such that the gaps in the self-cleaning web are wider at or about the at least one porous member than the gaps at or about the structural frame and/or the filter means.

In one embodiment, the method comprising cutting or otherwise forming gaps in the self-cleaning means corresponding to the size and position of the at least one porous member on or within the structural frame.

In one embodiment, the method comprises forming, placing and/or stretching the self-cleaning web over the structural frame and/or the at least one porous member.

In one embodiment, the method comprises applying an adhesive, clamp and/or mechanical fixing means to the self-cleaning means, the filter means and/or the structural means to retain the self-cleaning means, the filter means and/or the structural means together.

In another embodiment, the method comprises heating the semipermeable arrangement.

Ideally, the method comprises heating the self-cleaning means, the filter means and/or the structural means.

Preferably, the method comprises heating the self-cleaning means, the filter means and/or the structural means to at least the melting point of the filter means.

Preferably, the method comprises heating the self-cleaning means, the filter means and/or the structural means to at least the melting point of the polyurethane.

Preferably, the method comprises heating the self-cleaning means, the filter means and/or the structural means to at least 60° C.

Advantageously, this melts the filter means on to the structural means and/or the self-cleaning means and retains these components together.

Ideally, the method comprises cooling the semipermeable arrangement.

Preferably, the method comprises preparing at least one supplementary substance to be added to the semipermeable arrangement.

Ideally, the method comprises adding at least one supplementary substance to the structural means, the at least one porous member, the filter means and/or the self-cleaning means.

Preferably, the method comprises impregnating the structural means, the at least one porous member, the filter means and/or the self-cleaning means with at least one supplementary substance.

Ideally, the method comprises saturating the self-cleaning web and/or the structural means with at least one supplementary substance.

Preferably, the method comprises forming an envelope from the semipermeable arrangement, the envelope for use with a MAVIED.

Ideally, the method comprises forming two or more semipermeable arrangements and adjoining them.

Preferably, the method comprises adjoining two or more semipermeable arrangements to form an envelope for use with a MAVIED.

Preferably, the method comprises adjoining two or more semipermeable arrangements to form an envelope for use with a MAVIED by melting, clamping or other suitable means for adjoining.

Preferably, the method comprises adjoining two or more semipermeable arrangements to form an envelope for use with a MAVIED by applying a melted polymer to at least part of the two or more semipermeable arrangements.

Ideally, the method comprises fitting a reinforcement means to the envelope.

Preferably, the method comprises fitting a seal means to the envelope.

According to a fourth aspect of the invention there is provided a method of manufacture of a semipermeable arrangement for use in clinical, agricultural, industrial and/or environmental settings, the method comprising providing additives on or within a porous member.

Preferably, the method comprises providing additives on or within a plurality of porous members.

Preferably, the method comprises exposing the porous member to a solution containing one or more additives.

Ideally, the method comprises allowing the solvent of the solution to evaporate or actively encouraging evaporation through heating.

Preferably, the method comprises arranging the plurality of porous members within a structural means.

According to a fifth aspect of the invention there is provided an envelope for use with a MAVIED, the envelope being formed at least partially from at least one semipermeable arrangement, the at least one semipermeable arrangement comprising a structural means and at least one porous member, the at least one porous member being located on or within the structural means.

Advantageously, during implantation, the redundant lead length of the MAVIED can be coiled into a lead-top arrangement within the envelope without the risk of fibrosis or tissue in-growth over the lead.

Ideally, the envelope being sized to receive a pulse generator.

Preferably, the envelope comprising a substantially rectangular perimeter and/or a semicircular perimeter.

Ideally, the envelope comprises two spaced apart layers adjoined forming a side portion.

Ideally, the envelope comprises a cavity for receiving a pulse generator.

In one embodiment, the envelope comprises a single, folded semipermeable arrangement.

In another embodiment, the envelope comprises a plurality of semipermeable arrangements joined together.

Preferably, the envelope comprises a plurality of semipermeable arrangements joined together at least partially about their edges.

Advantageously, the semipermeable arrangements are self-cleaning and protect against microbial infection by inhibiting biofilm formation, as well as tissue ingrowth.

Ideally, the envelope comprises a seal means for sealing at least part of the envelope.

Preferably, the seal means comprises at least one sealing member.

Ideally, the at least one sealing member being arranged on the at least one semipermeable arrangement.

Ideally, the at least one sealing member being arranged extending along an edge portion of the at least one semipermeable arrangement.

Preferably, the seal means comprises two mutually opposing sealing members.

Ideally, the two mutually opposing sealing members being formed to engage with each other.

Ideally, the two mutually opposing sealing members being formed to engage with each other by pressing them together.

Preferably, the seal means comprises a male sealing member and a female sealing member.

Ideally, the female sealing member comprising an opening or groove sized to receive and retain the male sealing member in an interference fit.

Preferably, wherein the sealing members have been pressed together to form a seal, the sealing members can be pulled apart thereby releasing the seal.

Ideally, the seal means comprises a closure means for bringing the two sealing members into proximity with each other and at least partially sealing the envelope.

Ideally, the seal means being formed at least partially from PTFE.

Preferably, the seal means being formed at least partially from ePTFE.

Ideally, the at least one seal member being formed from PTFE, most preferably, ePTFE.

Ideally, the seal means comprises a reinforcement member.

Preferably, the reinforcement member holds the two mutually opposing sealing members together at a point along the length of the two mutually opposing sealing members.

Ideally, the reinforcement member holds the two mutually opposing sealing members together at or about the end of the two mutually opposing sealing members.

Ideally, the envelope comprises an exit point.

Preferably, the exit point is located at or about an end of the at least one sealing member.

Preferably, the exit point is sized to permit passage of a lead from inside the envelope to outside the envelope.

Ideally, the exit point is located at the side portion.

Advantageously, in use, the lead projects out from the side of the envelope and not the top or bottom. Furthermore, redundant lead length can be inserted into the envelope in a lead-top arrangement. The semipermeable arrangement protects the lead from tissue ingrowth, simplifying revision and extraction procedures.

Ideally, the envelope comprises an extended portion at or about the exit point.

Preferably, in use, the extended portion extends along a length of a lead.

Advantageously, a suture can be applied around the extended portion to seal the envelope.

Ideally, the envelope being compatible with lead anchor devices.

According to a sixth aspect of the invention there is provided a semipermeable arrangement for use with a MAVIED, the semipermeable arrangement being permeable to water and dissolved substances but impermeable to bacteria, the semipermeable arrangement being adaptable to allow passage of electrically charged particles through the semipermeable arrangement.

Ideally, the semipermeable arrangement comprising at least one porous member.

Preferably, the semipermeable arrangement comprising a structural means.

Ideally, the semipermeable arrangement comprising a self-cleaning means.

Preferably, the semipermeable arrangement comprising a means for joining two or more self-cleaning means together.

Preferably, the joining means comprises melted plastic, most preferably, polyurethane.

According to a seventh aspect of the invention there is provided a semipermeable arrangement for use in clinical, agricultural, industrial and/or environmental settings comprising a structural means, a filter means and/or a self-cleaning means.

According to an eighth aspect of the invention there is provided a semipermeable arrangement for use in clinical, agricultural, industrial and/or environmental settings comprising a structural means, the structural means being formed at least partially from a material having an affinity to a lubricating fluid and being adapted such that it can be infused with a lubricating fluid, the semipermeable arrangement being arranged with means to prevent or limit the movement of the lubricating fluid through at least part of the structural means.

Advantageously, when the lubricating fluid is applied to the structural means, the movement of lubricating fluid through the structural means is restricted, and there are areas on the semipermeable arrangement that are completely or substantially free from the presence of lubricating fluid.

Ideally, the structural means is porous.

Advantageously, the pores of the structural means can be infused with lubricating fluid or any fluid having an affinity to the material of the structural means. By applying a lubricating fluid, such as perfluorocarbon liquid, to the structural means, this prevents the likelihood of fouling occurring. However, it also renders the structural means impermeable to air, water and dissolved substances and it is therefore not applicable for some uses where a degree of permeability is required, such as when it is required for an ionic current to pass through the semipermeable arrangement (e.g. in MAVIED usage).

Ideally, the means to prevent or limit movement of the lubricating fluid through at least part of the structural means is arranged having one or more passageways to permit the movement of fluids such as air, water and dissolved substances through the structural means.

Preferably, the means to prevent or limit movement of the lubricating fluid through at least part of the structural means comprises at least one porous member.

Alternatively or additionally, the means to prevent or limit the movement of the lubricating fluid through at least part of the structural means comprises at least one substantially hollow member, the hollow member having a first opening arrangeable at one side of the structural means and a second opening arrangeable at another side of the structural means.

Ideally, the substantially hollow member is a tube.

Preferably, the substantially hollow member is formed at least partially from one or more polymeric substances, for example polyurethane (PU), fluorinated ethylene propylene (FEP) and/or PTFE.

Ideally, the substantially hollow member is formed at least partially from a relatively rigid polymeric substance such as PTFE.

Advantageously, this prevents the substantially hollow member from easily collapsing in use thereby blocking the passageway.

Ideally, the substantially hollow member is formed having a rigid innermost portion and less rigid outermost portion.

Preferably, the substantially hollow member comprises an outermost portion formed of a pliable substance or substance having a relatively low melting point (i.e. lower than that of PTFE) such as FEP or PU.

Advantageously, the outermost portion can be integrated into the structural means.

Ideally, the structural means is formed from one or more layers, most preferably, from one or more layers of ePTFE.

Preferably, the structural means comprises a first layer and a second layer of ePTFE.

Ideally, the first layer is arranged relative to the second layer such that the longitudinal direction of the fibrils in the first layer is misaligned with the longitudinal direction of the fibrils in the second layer.

Preferably, the first layer is arranged relative to the second layer such that the longitudinal direction of the fibrils in the first layer is oblique to the longitudinal direction of the fibrils in the second layer.

Ideally, the first layer is arranged relative to the second layer such that the longitudinal direction of the fibrils in the first layer extend parallel or orthogonal, or any angle therebetween, to the longitudinal direction of the fibrils in the second layer.

Most preferably, the first layer is arranged relative to the second layer such that the longitudinal direction of the fibrils in the first layer extend orthogonal to the longitudinal direction of the fibrils in the second layer.

The tensile strength of ePTFE varies depending on the direction of the force acting on the ePTFE, relative to the orientation of the ePTFE fibrils. By arranging the first and second layers such that the fibrils in the first layer run orthogonally to the fibrils in the second layer, the tensile strength of the structural means is uniform and cannot be easily deformed regardless of the direction of any disruptive force acting on the structural means.

Preferably, where the structural means is formed from a plurality of layers of ePTFE, each layer is bound together.

Expanded PTFE layers can be bound together by pressing the layers together. Alternatively, a binding polymer such as FEP can be placed between the layers and when the layers are pressed together the soft polymer fills gaps between fibrils in each layer and binds the layers together, thereby the binding polymer extends between the layers. Additionally or alternatively, heat can be applied to melt the binding polymer to bind the layers of the structural means together.

Ideally, the means to prevent or limit movement of the lubricating fluid through at least part of the structural means is retained within the structural means, most preferably, it is retained by the structural means.

Preferably, the means to prevent or limit movement of the lubricating fluid through at least part of the structural means is disposed between the first and second layers of the structural means.

Preferably, the means to prevent or limit movement of the lubricating fluid through at least part of the structural means extends out from a main plane of the structural means in at least one direction.

Advantageously, this provides a break in the plane of the structural means and the capillary action of perfluorocarbon liquid is disrupted when it reaches the means to prevent or limit movement of the lubricating fluid.

Ideally, the spacing between fibres in the structural means is equal to or less than 2.0 μm, most preferably equal to or less than 1.0 μm.

Ideally, the means to prevent or limit movement of the lubricating fluid through at least part of the structural means includes barriers located within the pores of the structural means.

Ideally, the barriers are provided by pressing and/or heat-melting a polymer such as PU or FEP into the gaps.

Advantageously, movement of the lubricating fluid is restricted by the presence of the barriers.

Preferably, the substantially hollow member has a flange adapted to be disposed between the first and second layers of the structural means.

Ideally, the flange can be heat-melted and/or pressed to fill the gaps between the fibrils of the structural means thereby providing a barrier to movement of lubricating fluid.

Ideally, the outermost portion of the substantially hollow member can be heat-melted and/or pressed to fill the gaps between the fibrils of the structural means thereby providing a barrier to movement of lubricating fluid.

Preferably, the outermost portion of the substantially hollow member is arranged such that it extends into the structural means thereby retaining the substantially hollow member within the structural means and providing a barrier to movement of lubricating fluid through the structural means.

Ideally, the structural means extends over the openings of the passageways of the means to prevent or limit movement of the lubricating fluid through at least part of the structural means.

Advantageously, the barriers to movement of the lubricating fluid, as provided by the substantially hollow member or otherwise, prevent the lubricating fluid from extending through the portion of the structural means that extends over the passageways. As the structural means is porous, this permits movement of fluids such as air and water through the passageways. In one embodiment, the structural means further acts as a filter to prevent movement of bacteria through the passageways. When impregnated with lubricating fluid, the semipermeable arrangement is thereby both self-cleaning (as provided by the lubricant) and permeable to air or water, but also impermeable to particles and bacteria sized greater than the minimum pore size of the structural means.

Preferably, the semipermeable arrangement, most preferably the structural means, comprises lubricating fluid, most preferably, the lubricating fluid is perfluorocarbon liquid.

Preferably, at least part of, most preferably the passageways of, the means to prevent or limit movement of the lubricating fluid through at least part of the structural means comprises additives, non-limiting examples of possible additives include soluble particles of one or more chemical substances and/or a water- and/or air-purifying substance such as activated charcoal.

Ideally, the portion of the structural means that extends over the passageways of the means to prevent or limit the movement of the lubricating fluid through at least part of the structural means is at least partially hydrophilic.

Advantageously, water is not repelled from the portion of the structural means that extends over the passageways and so water can move through the passageways. The structural means that extends over the passageway can be chemically modified PTFE (e.g. with the addition of hydroxyl groups), heat-treated PTFE, or formed from an alternative polymeric, hydrophilic substance such as hydrophilic PU.

Ideally, the semipermeable arrangement is adaptable or adapted for implantation into a recipient, for use with a MAVIED, for use as a wound dressing, for use as a face mask, for use as a water filter, or for use as an air filter.

Advantageously, the semipermeable arrangement is both self-cleaning and permeable and therefore has a wide range of uses. As a wound dressing, the semipermeable arrangement prohibits bacteria from accessing the wound but is breathable and allows air to access the wound. Furthermore, if infused with a lubricating fluid, the recipient's cells will not adhere to the dressing. This is a common problem with traditional dressings whereby newly formed cells are removed when the dressing is changed as the cells come away with the dressing. Many known face masks have a mesh size that is too large to prohibit movement of bacteria through the mask, but the mesh size cannot be made smaller as this would restrict the user's breathing. Known masks are also prone to fouling and can thereby act as a source of infection. The semipermeable arrangement allows movement of air therethrough but not bacteria and resists fouling when infused with lubricating fluid and is therefore advantageous over known face masks. Water filters must be changed regularly, disposing of the old filter and replacing with a new, clean filter. The semipermeable arrangement resists fouling and so can easily be wiped clean when required. When used as a water filter, the filter has a longer period of use than prior art water filters. Likewise, a similar problem occurs in air filters whereby the filter occasionally must be replaced, whereas the semipermeable arrangement of the current application, if used as an air filter, could be easily cleaned instead of being disposed of.

According to a ninth aspect of the invention there is provided a method of manufacturing a semipermeable arrangement for use in clinical, agricultural, industrial and/or environmental settings, the method comprising forming a structural means that has an affinity to a lubricating fluid is adapted such that it can be infused with a lubricating fluid, the method further comprising arranging a means to prevent or limit movement of the lubricating fluid through at least part of the structural means.

Ideally, the method comprises forming the structural means such that it is porous or forming the structural means from a porous material.

Preferably, the method comprises providing or forming a means to prevent or limit movement of the lubricating fluid through at least part of the structural means, whereby the means is arranged having one or more passageways to permit the movement of fluids such as air, water and dissolved substances through the structural means.

In one embodiment the method involves providing at least one porous member adapted to prevent or limit movement of the lubricating fluid through the structural means.

Alternatively or additionally, the method involves forming or providing a substantially hollow member, for example a tube, adaptable to prevent or limit movement of the lubricating fluid through the structural means, wherein the substantially hollow member has a first opening arrangeable at one side of the structural means and a second opening arrangeable at another side of the structural means.

Ideally, the method comprises forming the substantially hollow member from one or more polymeric substances, for example polyurethane (PU), fluorinated ethylene propylene (FEP) and/or PTFE.

Preferably, the method comprises forming the substantially hollow member having a rigid inner wall and a pliable outer wall.

Ideally, the method comprises forming the substantially hollow member having an inner wall formed from PTFE and/or an outer wall formed from PU or FEP.

Preferably, the method comprises forming the substantially hollow member with one or more flanges that can be integrated between layers of the structural means.

Ideally, the method comprises forming the semipermeable arrangement by first providing a first layer of the structural means, the first layer having fibrils extending in a first direction.

Preferably, the method comprising placing one or more porous members and/or substantially hollow members on the first layer.

Ideally, the method comprising placing a second layer of the structural means, the second layer having fibrils extending in a second direction, over the one or more porous members and/or substantially hollow members.

Preferably, the method comprising arranging the first layer relative to the second layer such that the second direction is parallel or orthogonal to the first direction or at an angle between parallel and orthogonal.

Most preferably, the method comprising arranging the first layer relative to the second layer such that the second direction is orthogonal to the first direction.

Ideally, the method comprising sealing the first and second layers together.

Preferably, the method comprising sealing the first and second layers together by pressing them together.

Preferably, the method comprising sealing the first and second layers together by placing a material such as FEP or UP that is pliable or can be melted between the first and second layers, and pressing or heat-treating the structural means.

Preferably, the method comprising pressing or heat-treating the structural means at or about the substantially hollow member.

Ideally, the method comprises providing a porous and/or hydrophilic portion of the structural means extending over the openings of the passageways of the means to prevent or limit movement of the lubricating fluid through at least part of the structural means.

Preferably, the method comprises infusing the structural means with a lubricating fluid, most preferably, with perfluorocarbon liquid.

Ideally, the method comprises disposing additives, non-limiting examples include soluble particles of one or more chemical substances and/or activated charcoal within the passageways of, the means to prevent or limit movement of the lubricating fluid through at least part of the structural means.

Ideally, the method comprises adapting the semipermeable arrangement for implantation into a recipient, for use with a MAVIED, for use as a wound dressing, for use as a face mask, for use as a water filter, or for use as an air filter.

According to a tenth aspect of the invention there is provided an envelope for use with a MAVIED, the envelope being formed at least partially from at least one semipermeable arrangement, the at least one semipermeable arrangement comprising a structural means, the structural means being formed at least partially from a material having an affinity to a lubricating fluid and being adapted such that it can be infused with a lubricating fluid, the semipermeable arrangement being arranged with means to prevent or limit the movement of the lubricating fluid through at least part of the structural means.

It will be appreciated that optional features applicable to one aspect of the invention can be used in any combination, and in any number. Moreover, they can also be used with any of the other aspects of the invention in any combination and in any number. This includes, but is not limited to, the dependent claims from any claim being used as dependent claims for any other claim in the claims of this application.

The invention will now be described with reference to the accompanying drawings which shows by way of example only twelve embodiments of an apparatus in accordance with the invention.

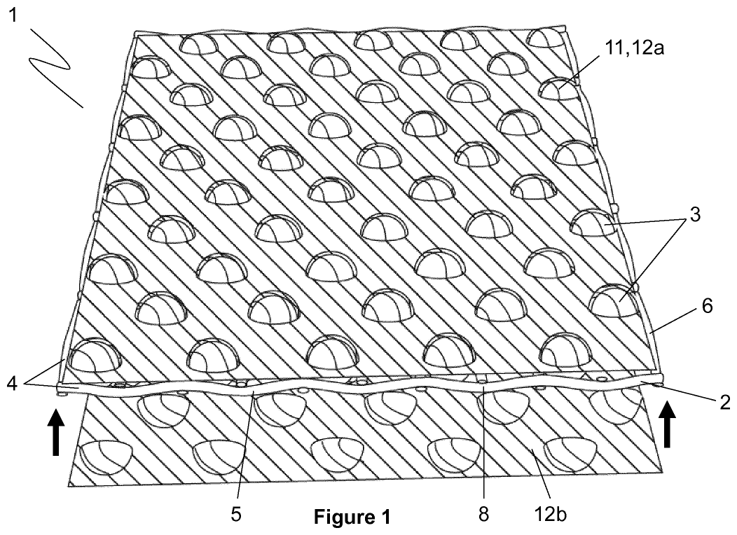

FIG. 1 is a perspective view of a first embodiment of a semipermeable arrangement according to the invention wherein no lubricant is present. The semipermeable arrangement is shown in expanded view with the lower filter web located away from the structural frame with arrows indicating the assembly direction.

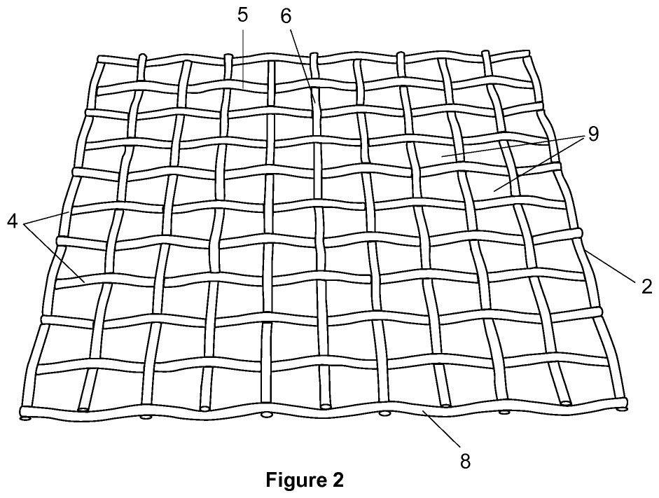

FIG. 2 is a perspective view of the structural frame of the semipermeable arrangement of FIG. 1.

FIG. 3 is a front elevation view of a first embodiment of a porous member of the semipermeable arrangement of FIG. 1.

FIG. 4 is a perspective view of a second embodiment of a porous member.

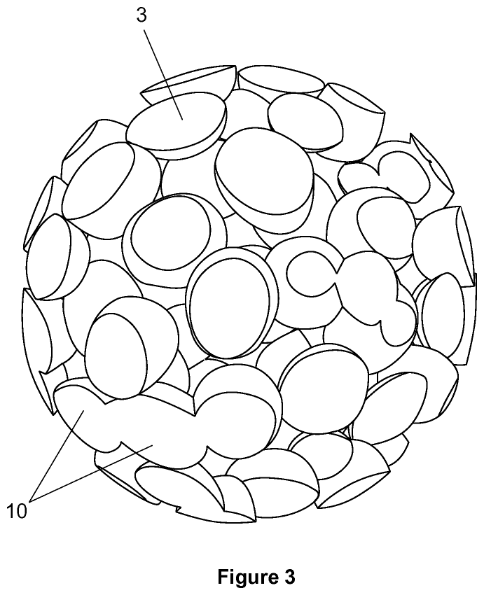

FIG. 5 is a perspective view of a third embodiment of a porous member.

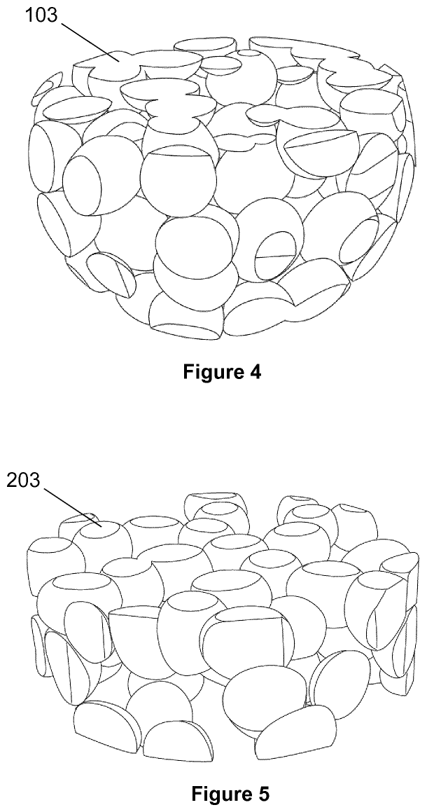

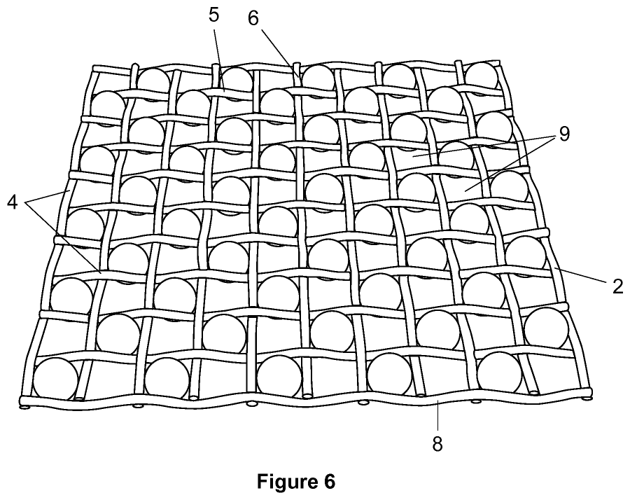

FIG. 6 is a perspective view of the structural frame and the plurality of porous members of the semipermeable arrangement of FIG. 1.

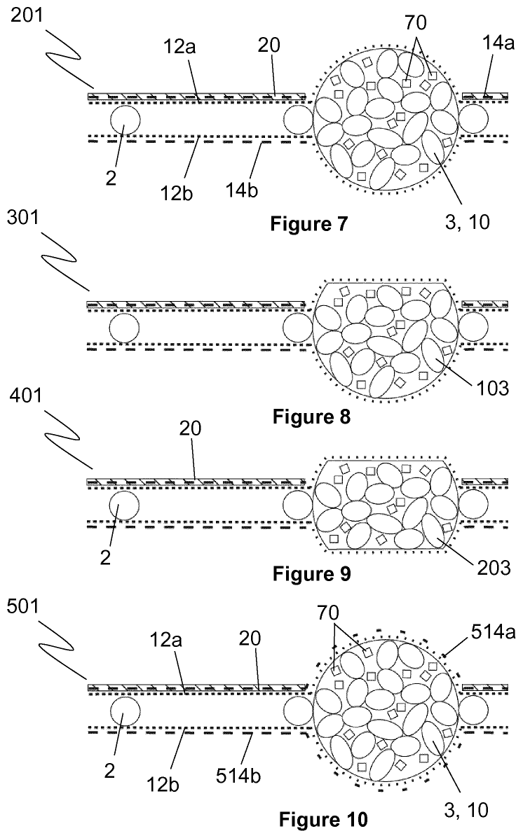

FIG. 7 is a cross-sectional view of a second embodiment of a semipermeable arrangement according to the invention wherein the self-cleaning web is not extended over the surface of the porous member and wherein the lubricant is present on one side of the semipermeable arrangement, further illustrating crystallized antimicrobial particles within the porous member.

FIG. 8 is a cross-sectional view of a third embodiment of a semipermeable arrangement according to the invention wherein the porous member is a spherical cap.

FIG. 9 is a cross-sectional view of a fourth embodiment of a semipermeable arrangement according to the invention wherein the porous member is disc-shaped.

FIG. 10 is a cross-section view of a fifth embodiment of a semipermeable arrangement according to the invention wherein the self-cleaning web is extended over the surface of the porous member and wherein the lubricant is present on one side of the semipermeable arrangement.

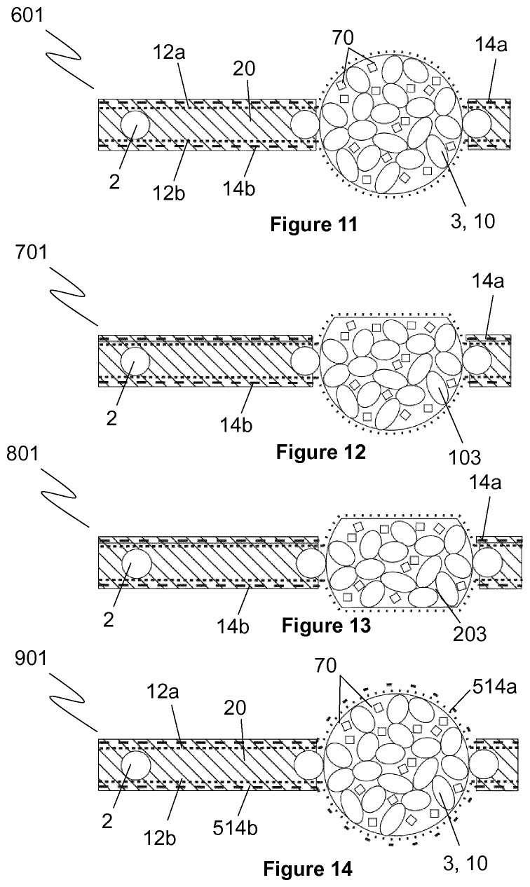

FIG. 11 is a cross-sectional view of a sixth embodiment of a semipermeable arrangement according to the invention wherein the self-cleaning web is not extended over the surface of the porous member and wherein the lubricant is present on both sides of the semipermeable arrangement and throughout the structural frame.

FIG. 12 is a cross-sectional view of a seventh embodiment of a semipermeable arrangement according to the invention wherein the porous member is a spherical cap.

FIG. 13 is a cross-sectional view of a eighth embodiment of a semipermeable arrangement according to the invention wherein the porous member is disc-shaped.

FIG. 14 is a cross-sectional view of a ninth embodiment of a semipermeable arrangement according to the invention wherein the self-cleaning web is extended over the surface of the porous member and wherein the lubricant is present on both sides of the semipermeable arrangement and throughout the structural frame.

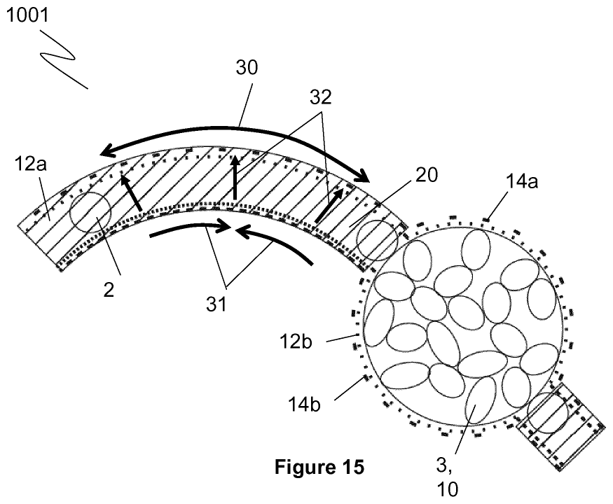

FIG. 15 is a cross-sectional view of a tenth embodiment of a semipermeable arrangement according to the invention wherein the semipermeable arrangement is bent, showing redistribution of the lubricant through the structural frame.

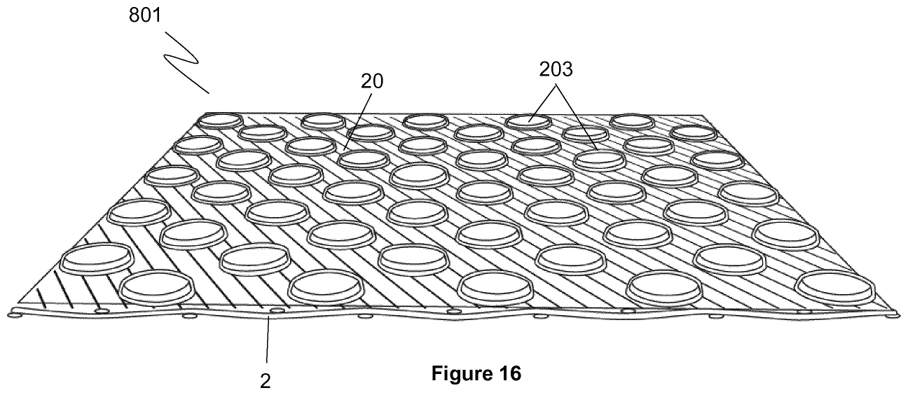

FIG. 16 is a perspective view of a semipermeable arrangement having disc-shaped porous members.

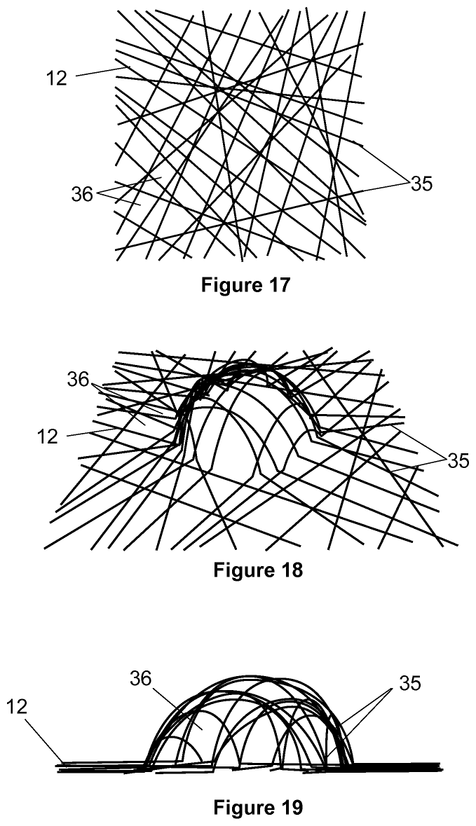

FIG. 17 is a plan view of a filter web according to the invention as it would appear when used in an embodiment of the invention comprising spherical or hemispherical porous members.

FIG. 18 is a perspective view of the filter web in FIG. 17.

FIG. 19 is a side elevation view of the filter web in FIG. 17.

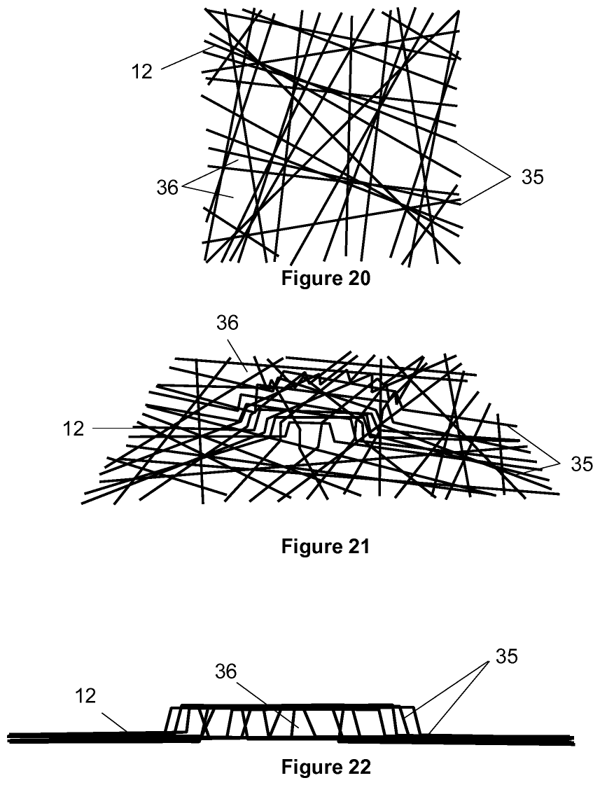

FIG. 20 is a plan view of a filter web according to the invention as it would appear when used in an embodiment of the invention comprising disc-shaped porous members.

FIG. 21 is a perspective view of the filter web in FIG. 20.

FIG. 22 is a side elevation view of the filter web in FIG. 20.

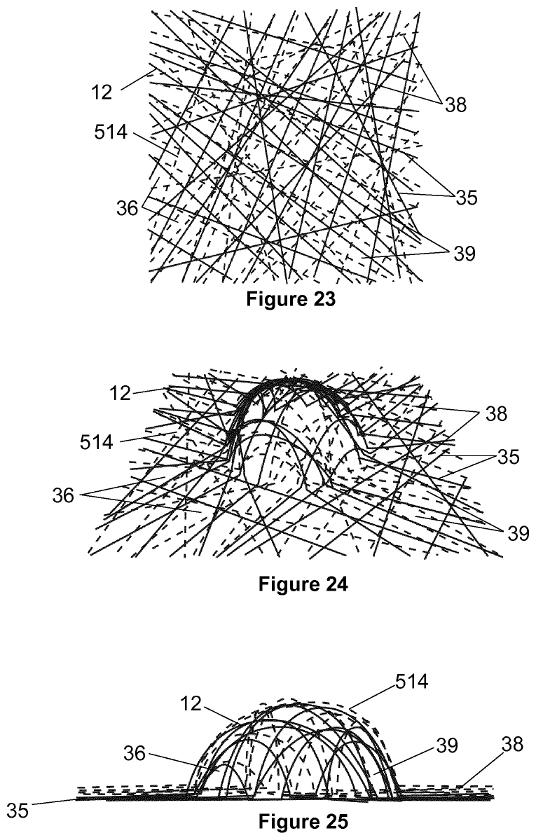

FIG. 23 is a plan view of the filter web and the self-cleaning web arranged on top of the filter web according to the invention and as present in the fifth and ninth embodiments of the semipermeable arrangement shown in FIGS. 10 and 14 respectively. The self-cleaning web is illustrated as dashed lines.

FIG. 24 is a perspective view of the filter web and self-cleaning web of FIG. 23.

FIG. 25 is a side elevation view of the filter web and self-cleaning web of FIG. 23.

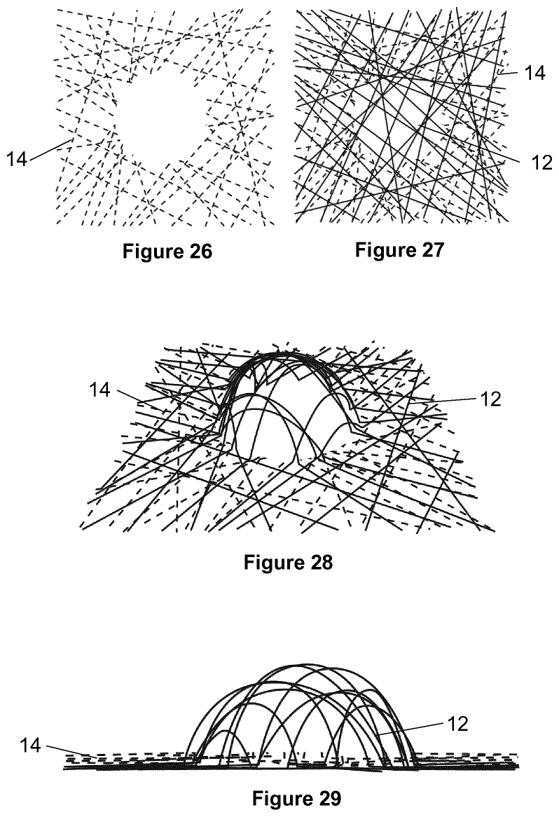

FIG. 26 is a plan view of a self-cleaning web according to the invention.

FIG. 27 is a plan view of the self-cleaning web shown in FIG. 26 overlaying a filter web according to the invention and as present in the second and sixth embodiments of the semipermeable arrangement shown in FIGS. 7 and 11 respectively.

FIG. 28 is a perspective view of the self-cleaning web and filter web of FIG. 27.

FIG. 29 is a side-elevation view of the self-cleaning web and filter web of FIG. 27.

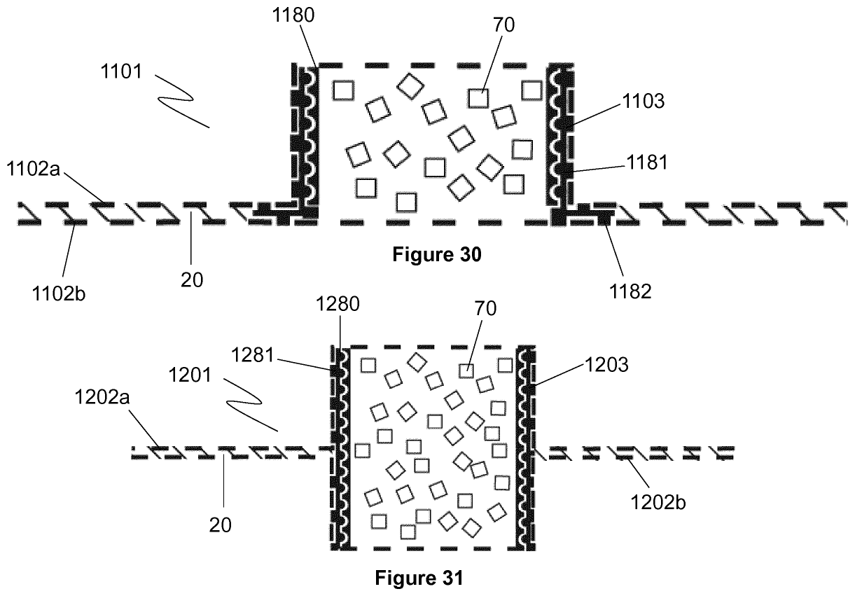

FIG. 30 is a side elevation, cross sectional view of a semipermeable arrangement according to an eleventh embodiment of the invention.

FIG. 31 is a side elevation, cross sectional view of a twelfth embodiment of a semipermeable arrangement according to the invention.

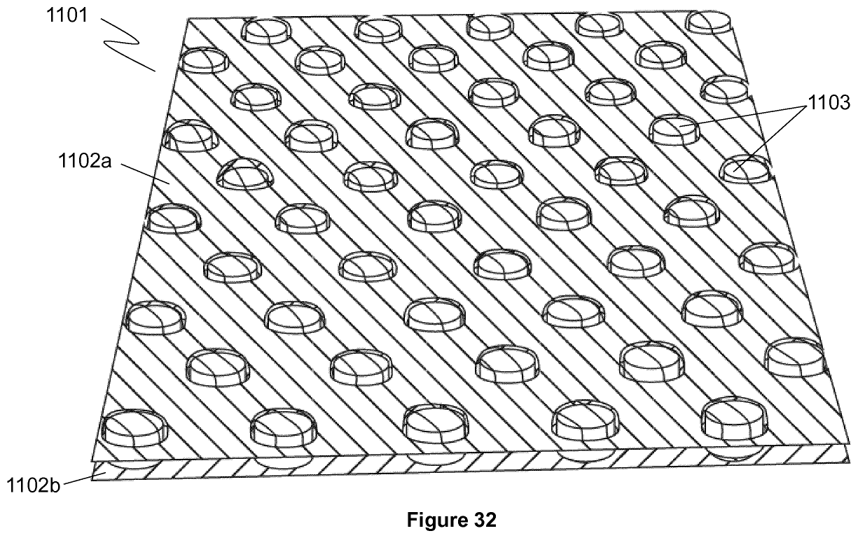

FIG. 32 is a perspective view the eleventh embodiment as shown in FIG. 30.

FIG. 33 is a rear perspective view of an envelope formed from a semipermeable arrangement for use with a MAVIED according to the invention, porous members have not been illustrated for clarity purposes.

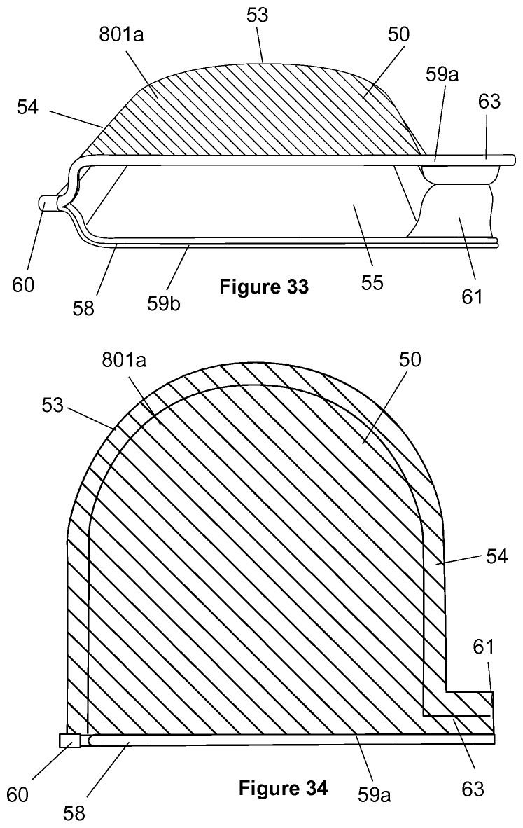

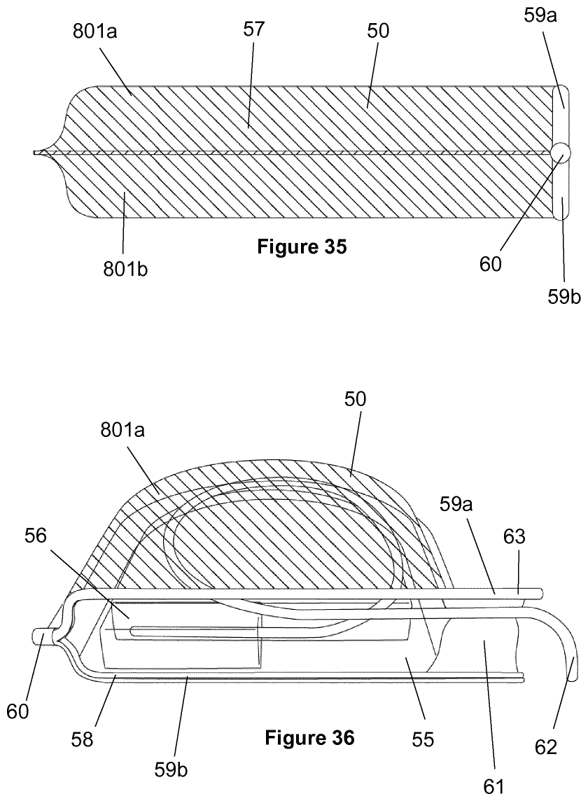

FIG. 34 is a plan view of the envelope in FIG. 33.

FIG. 35 is a side elevation view of the envelope in FIG. 33.

FIG. 36 is a rear perspective view of the envelope in FIG. 33 in use containing a pulse generator, not sealed. The envelope is shown in low opacity for illustration purposes.

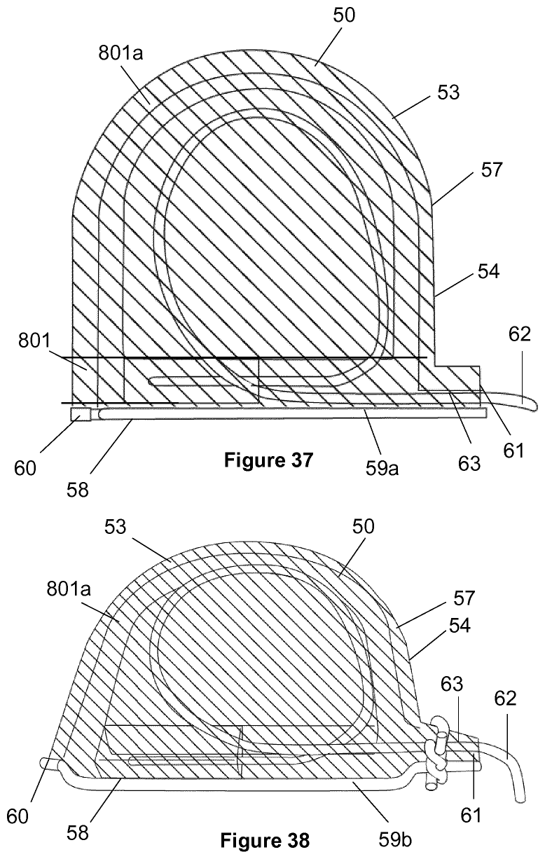

FIG. 37 is a plan view of the envelope as shown in FIG. 33.

FIG. 38 is a rear perspective view of the envelope in FIG. 33 after it has been sealed and a suture applied to the exit portion.

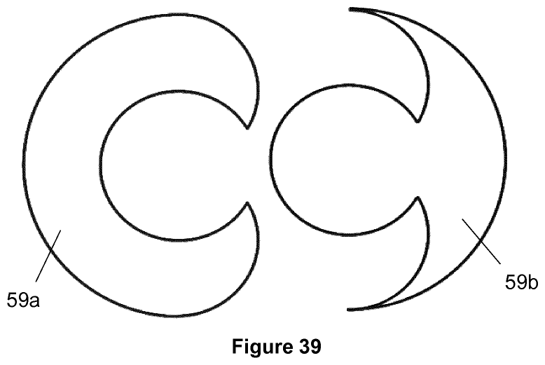

FIG. 39 is a cross section of sealing members according to the invention.

In the drawings there is a shown a semipermeable arrangement according to the invention. FIGS. 1 to 3 show a first embodiment of a semipermeable arrangement and components thereof indicated generally by reference numeral 1. The semipermeable arrangement has a structural frame 2 and a plurality of porous members 3 that are arranged within the structural frame 2. The structural frame 2 is a single layered frame formed from a plurality of structural rods 4. The structural rods 4 are arranged as a first group of structural rods and a second group of structure rods 6, wherein each group of structural rods 5, 6 have a plurality of structural rods arranged in a spaced apart, parallel relationship. The spacing between the structural rods 4 in the first group of structural rods 5 and the second group of structural rods 6 is 3 mm. The first group of structural rods 5 is arranged perpendicular to the second group of structural rods 6 and the they are woven together to form a mesh network 8 with square-shaped openings 9 having an area of around 9 mm² each (see FIG. 2). The structural rods 2 have a circular cross-section and a diameter of around 1 mm and are formed from ePTFE.

FIG. 3 shows an expanded view of a porous member 3. The porous members 3 are roughly spherical and have a diameter of around 3 mm enabling them to be located in the openings 9 within the mesh network 8. When they are located in openings 9 of the mesh network 8 the structural rods 4 press against, hold and retain the porous members 3 in the structural frame. The porous members 3 are formed from polypropylene and are manufactured by sintering together small, loosely compacted particles 10 of polypropylene. This provides gaps between the particles which act as pores to permit passage of water and dissolved substances into and through the porous members 3. The pore size of the porous members 3 is varied but they are less than or equal to 50 μm. The porous members 3 are arranged in the structural frame 3 and extend out of the plane of the structural frame by 1 mm each side of the structural frame.

FIG. 4 shows an alternative porous member indicated by reference numeral 103. The porous member 103 is a truncated sphere or spherical cap. The porous member 103 can be formed, for example, in the same way as the spherical porous member 3 and then removing a portion. The porous member 103 is arranged in a structural frame with the plane of the flat surface of the porous member 103 being parallel with the plane of the frame, and the rounded surface extending beyond the plane of the structural frame at the opposing surface of the frame. The flat, circular surface has a radius of 1.4 mm.

FIG. 5 shows a further alternative porous member indicated by reference numeral 203. The porous member 203 is disc-shaped and can be formed, for example, by producing a spherical porous member as in FIG. 3 and removing two portions, or by producing a porous sheet and punching out round porous members having a radius of 1.5 mm and a thickness of 1 mm. When arranged in a structural frame, the plane of the flat surfaces are parallel with the plane of the structural frame.

As shown in FIG. 6, the plurality of porous members 3 are located interspersed within the structural frame 2 in rows within the structural frame 2 such that every other opening in the row of the mesh network 8 of the structural frame 2 contains a porous member 3 and that a row above or below this row contains a similar pattern but is offset relative to the row such that the plurality of porous members 3 are arranged in a diagonal relationship within the mesh network 8 but not laterally or longitudinally adjacent to one another.

The semipermeable arrangement 1 further has a filter arrangement 11 having two filter webs 12a, 12b with spacings (see FIG. 1). The filter webs 12a, 12b are formed by electrospinning polyurethane to form filter web fibres that are irregularly arranged, overlapping to and have spacings therebetween. The spacings are equal to or less than equal to or less than 0.2 μm, thereby filtering out any matter with a diameter larger than 0.2 μm. The filter webs 12a, 12b are arranged on the surface of the structural frame 2 and the porous members 3 and are stretched tight over them. The filter arrangement 11 is arranged on both sides of the structural frame 2 creating a bilayer effect. FIG. 1 shows an expanded view of the semipermeable arrangement 1 wherein the lower filter web 12b is located away from the structural frame 2 for illustration purposes.

In use, the semipermeable arrangement 1 can be used as a filter membrane in clinical, agricultural, industrial or environmental settings. For example, the semipermeable arrangement 1 can be positioned over a vessel and a fluid to be filtered can be added to one side of the semipermeable arrangement 1. The pores of the filter webs 12a, 12b are sized less than 1 μm so any matter greater than this will be retained on one side of the semipermeable arrangement 1 as the fluid filters through the semipermeable arrangement 1. The semipermeable arrangement 1 can be easily removed and cleaned and the porous members 3 provide a roughened texture to the surface of the semipermeable arrangement 1, preventing matter from settling across the surface of the semipermeable arrangement 1.

In the second embodiment of the invention illustrated in FIG. 7 there is shown a semipermeable arrangement indicated generally by reference numeral 201 having a structural frame 2 with a porous member 3 formed from particles 10. The semipermeable arrangement 201 further has soluble particles 70 of an antimicrobial located within the porous member 3. The semipermeable arrangement 201 further has two filter webs 12a, 12b and two self-cleaning webs 14a, 14b arranged at each side of the structural frame 2 to create a bilayer effect. The self-cleaning webs 14a, 14b are formed from electrospinning PTFE to form self-cleaning web fibres that are irregularly arranged, overlapping and have spacings therebetween. The self-cleaning webs 14a, 14b are adapted, through control of the spacings between the self-cleaning web fibres, to receive and uptake a lubricant via capillary action. The semipermeable arrangement 201 further has perfluorocarbon liquid 20 at one surface of the semipermeable arrangement 201. The perfluorocarbon liquid 20 is dispersed through the self-cleaning web 14a. The self-cleaning web 14a is formed from PTFE and so the perfluorocarbon liquid 20 has a natural affinity for the self-cleaning web 14a. In addition, the spacings of the self-cleaning web 14a are sized to allow capillary uptake of the perfluorocarbon liquid 20, dispersing it over the surface of the semipermeable arrangement 201. The filter web 12a, however, does not contain any fluorine atoms and the ionophilicity of the perfluorocarbon liquid prevents it from dispersing through the filter web 12a. The self-cleaning web 14a contains gaps at the location of the porous member 3. Therefore, none of the perfluorocarbon liquid 20 extends over the surface of the porous member 3. Water and dissolved substances can enter and pass through the filter webs 12a, 12b and the porous member 3 even when the perfluorocarbon liquid 20 is present within the self-cleaning web 14a.

In use, the semipermeable arrangement 201 can be used as a filter membrane in clinical, agricultural, industrial or environmental settings. For example, the semipermeable arrangement 201 can be positioned over a vessel with self-cleaning web 14a containing the perfluorocarbon liquid 20 facing out of the vessel. A fluid to be filtered can be added to this side of the semipermeable arrangement 201. The filter web 12a acts as a barrier to prevent matter sized less than 1 μm entering the semipermeable arrangement 201 and the omniphobicity of the perfluorocarbon liquid 20 repels the majority of substances from the surface of semipermeable arrangement 201; for example, hydrophilic and hydrophobic substances that contribute to fouling.

In the third embodiment of the invention illustrated in FIG. 8 there is shown a semipermeable arrangement indicated generally by reference numeral 301. The third embodiment 301 is similar to the second embodiment 201 but in that the porous member is a spherical cap porous member 103. In use, the surface of the semipermeable arrangement 301 with the flat surface of the porous members 103 can be orientated to abut a flat surface, such as that of a MAVIED, with the rounded portion of the porous members 103 arranged projecting out from the flat surface. The rounded portions help prevent fouling whereas the flat portion allows the semipermeable arrangement 301 to sit neat against the surface and can help anchor the semipermeable arrangement 301 to a surface. In addition, the porous member 103 has a larger internal void volume than the disc-shaped porous member 203. However, it may not be as technically straightforward as the disc-shaped porous member 203 to manufacture, which can simply be punched out of a porous sheet.

In the fourth embodiment of the invention illustrated in FIG. 9 there is shown a semipermeable arrangement indicated generally by reference number 401. The fourth embodiment 401 is similar to the second embodiment 201 but in that the porous member is a disc-shaped porous member 203. In use, both planar surfaces of the semipermeable arrangement 401 are identical with the exception that one surface has a layer of perfluorocarbon 20. Both planar surfaces of the porous member 203 are almost flush and just slightly outset from the planar surfaces of the structural frame 2. The thickness of the semipermeable arrangement 401 is close to being equal to the thickness of the structural frame 2.

In the fifth embodiment of the invention illustrated in FIG. 10 there is shown a semipermeable arrangement indicated generally by reference numeral 501 having a structural frame 2 with a porous member 3 formed from particles 10. The semipermeable arrangement 501 further has soluble particles 70 of an antimicrobial located within the porous member 3. The semipermeable arrangement 501 further has two filter webs 12a, 12b and two self-cleaning webs 514a, 514b arranged at each side of the structural frame 2 to create a bilayer effect. The semipermeable arrangement 501 further has a perfluorocarbon liquid 20 at one surface of the semipermeable arrangement 501. The perfluorocarbon liquid 20 is dispersed through the self-cleaning web 514a. In this embodiment, the self-cleaning webs 514a, 514b extend over the porous member 3. As a result of stretching, the spacings between the self-cleaning webs 514a, 514b are greater around the porous member 3 than the spacings between the self-cleaning webs 14a, 14b located at an opening of the structural frame 20. The increase in spacings of the self-cleaning webs 14a, 14b at the porous member 3 prevents dispersion of the perfluorocarbon liquid 20 through the self-cleaning webs 514a, 514b at the porous member 3 by capillary action. Therefore, none of the perfluorocarbon liquid 20 extends over the surface of the porous member 3. Water and dissolved substances can enter and pass through the filter webs 12a, 12b, the self-cleaning webs 514a, 514b and the porous member 3 even when the perfluorocarbon liquid is present within the self-cleaning web 514a.

In use, the semipermeable arrangement 501 can be used as a filter membrane in clinical, agricultural, industrial or environmental settings. For example, the semipermeable arrangement 501 can be positioned over a vessel with self-cleaning web 14a containing the perfluorocarbon liquid 20 facing out of the vessel. A fluid to be filtered can be added to this side of the semipermeable arrangement 501. The filter web 12a and the self-cleaning web 514a acts as a physical barrier to prevent matter sized less than 1 μm entering the semipermeable arrangement 501 and the omniphobicity of the perfluorocarbon liquid 20 repels the majority of substances from the surface of semipermeable arrangement 501.

In the sixth embodiment of the invention illustrated in FIG. 11 there is shown a semipermeable arrangement indicated generally by reference numeral 601. The structural frame 2, filter webs 12a, 12b, self-cleaning webs 14a, 14b and porous member 3 are the same as that of the second embodiment 201 (FIG. 7). The sixth embodiment 601 differs in that the perfluorocarbon liquid 20 is located throughout the structural frame 2 and is present on both sides of the semipermeable arrangement 601 and throughout both self-cleaning webs 14a, 14b. The self-cleaning webs 14a, 14b contains gaps at the location of the porous member 3 so none of the perfluorocarbon liquid 20 extends over the surface of the porous member 3. The structural frame 2 acts as a reservoir to replenish perfluorocarbon liquid 20 at the surfaces of the semipermeable arrangement 601 when the perfluorocarbon liquid 20 is depleted.

In use, the semipermeable arrangement 601 can be used as a filter membrane in clinical, agricultural, industrial or environmental settings. For example, the semipermeable arrangement 601 can be positioned over a vessel with either the self-cleaning web 14a or self-cleaning web 14b facing out of the vessel. A fluid to be filtered can be added the surface of the semipermeable arrangement 601. The filter webs 12a or 12b act as a physical barrier to prevent matter sized less than 1 μm entering the semipermeable arrangement 301 and the omniphobicity of the perfluorocarbon liquid 20 repels all substances lacking an affinity to perfluorocarbon from the surfaces of the semipermeable arrangement 601.

In the seventh embodiment of the invention illustrated in FIG. 12 there is shown a semipermeable arrangement indicated generally by reference numeral 701. The seventh embodiment 701 is similar to the third embodiment 301 (FIG. 8) but in that the perfluorocarbon liquid 20 is located throughout the structural frame 2 and is present on both sides of the semipermeable arrangement 701 and throughout both self-cleaning webs 14a, 14b.

In the eighth embodiment of the invention illustrated in FIG. 13 there is shown a semipermeable arrangement indicated generally by reference number 801. The eighth embodiment 801 is similar to the fourth embodiment 401 (FIG. 9) but in that the perfluorocarbon liquid 20 is located throughout the structural frame 2 and is present on both sides of the semipermeable arrangement 801 and throughout both self-cleaning webs 14a, 14b. In use, the semipermeable arrangement 801 has self-cleaning properties on both planar surfaces and as such it may be orientated in either way, where self-cleaning is desired on both surfaces. The eight embodiment is further illustrated in FIG. 16.

In the ninth embodiment shown in FIG. 14 there is shown a semipermeable arrangement indicated generally by reference numeral 901. The semipermeable arrangement 901 is similar to that of the fifth embodiment 501 shown in FIG. 10, but differs in that the perfluorocabon liquid 20 extends throughout structural frame 2 and is present on both sides of the semipermeable arrangement 801 and throughout both self-cleaning webs 14a, 14b. In use, the semipermeable arrangement 901 can be used as a filter membrane in clinical, agricultural, industrial or environmental settings. For example, the semipermeable arrangement 901 can be positioned over a vessel with either the self-cleaning web 14a or self-cleaning web 14b facing out of the vessel. A fluid to be filtered can be added the surface of the semipermeable arrangement 901. The filter webs 12a or 12b and the self-cleaning webs 14a or 14b act as a physical barrier to prevent matter sized less than 1 μm entering the semipermeable arrangement 901 and the omniphobicity of the perfluorocarbon liquid 20 repels the majority of substances from the surface of semipermeable arrangement 901.

In the tenth embodiment of the invention illustrated in FIG. 15, there is shown a semipermeable arrangement 1001 with a structural frame 2 and perfluorocarbon liquid 20. The tenth embodiment is similar to the ninth embodiment (FIG. 14) but differs in that there are no soluble antimicrobial particles 70. As shown, when the structural frame 2 is bent the perfluorocarbon liquid is redistributed. The outer perimeter of the curve of the semipermeable arrangement 1001 is stretched in the direction of the arrow 30 whereas the inner perimeter of the curve contracts in the direction of the arrows 31. The contraction at the inner perimeter of the curve reduces the volume at this portion of the structural frame and forces redistribution of the perfluorocarbon liquid 20, in the direction of the arrows 32, to the outer perimeter of the curve, thus ensuring that the outer perimeter of the curve remains lubricated by the perfluorocarbon 20 even when the structural frame 20 is bent.

FIGS. 17 to 19 illustrate a filter web 12 according to the invention as it would appear in situ over a spherical or hemispherical porous member 3 for illustration purposes. The filter web 12 comprises a plurality of filter web fibres 35 formed from electrospun polyurethane with gaps defining pores 36. The pores are no greater than 1 μm in size and therefore prevent passage of matter with dimensions greater than 1 μm from passing through the filter web 12. FIGS. 20 to 22 illustrate the filter web 12 according to the invention as it would appear in situ over a disc-shaped porous member 203 for illustration purposes.

FIGS. 23 to 25 illustrate a filter web 12 and a self-cleaning web 514 according to the invention as it would appear in situ over a porous member 3 for illustration purposes. In this arrangement, the self-cleaning web 514 extends over the filter web 12 in accordance with the first, fifth, ninth and tenth embodiments of the invention. The self-cleaning web 514 is formed from self-cleaning web fibres 38, which are formed from electrospun PTFE having pores 39. The pore size of the self-cleaning 514 is greater around the porous member and this prohibits uptake of a lubricant via capillary action, leaving the surface of the porous member free for passage of water and dissolved substances. FIGS. 26 to 29 illustrate a filter web 12 and a self-cleaning web 14 according to the invention as it would appear in situ over a porous member 3 for illustration purposes. In this arrangement, the self-cleaning web 14 has gaps sized to accommodate a porous member in accordance with the second, third, fourth, sixth, seventh and eighth embodiments of the invention.

FIGS. 30 and 32 illustrate an eleventh embodiment of the invention indicated by reference numeral 1101, and FIG. 32 shows a twelfth embodiment of the invention indicated by reference numeral 1201. Each of these semipermeable arrangements 1101, 1201 have a structural arrangement 1102, 1202 formed from a first layer 1102a, 1202a and a second layer 1102b, 1202b of ePTFE, pressed together. As illustrated in FIG. 32, the longitudinal direction of the fibrils—represented by solid black lines—in the first layer 1102a is orthogonal to that of the second layer 1102b and this provides a structural arrangement 1102 of uniform tensile strength. The spacings between fibrils of the ePTFE is equal to or less than 0.5 μm such that pathogenic bacteria are excluded from moving through the first or second layers 1102a, 1202a,1102b, 1202b. The semipermeable arrangements 1101, 1201 have a plurality of tubes 1103, 1203 that extend between the first layer 1102a, 1202a and the second layer 1102b, 1202b. The tubes 1103, 1203 provide a barrier to movement of lubricating fluid through the structural arrangement 1102, 1202 whilst permitting the movement of fluids such as air, water and dissolved substances across the structural arrangement 1102, 1202 via the interior of the tubes 1103, 1203.

Each tube 1103, 1203 is formed from an interior cylinder of PTFE 1180, 1280 and a coating of FEP 1181, 1281. In the eleventh embodiment, the tubes 1103 each have a flange 1182 that extends around the base of the tube 1103 and is sized to fit between the first layer 1102a and second layer 1102b of the structural arrangement 1102. During manufacture, the FEP coating 1181, 1281 extends into spacings between fibrils of the structural arrangement 1102 and this provides a barrier to movement of lubricating fluid through the structural arrangement 1102. The FEP coating can be extending into the spacings by pressing the first and second layers 1102a, 1102b into the FEP or by applying heat to melt the FEP into the spacings. Alternative materials to FEP may be used provided they can fill the gaps between fibrils of PTFE and create a barrier to lubricating fluid. The tubes 1103 of the eleventh embodiment are sized having a diameter of 3 mm and a height of 1 mm and are arranged to extend out of main plane of the first layer 1102a only. The second layer 1102b is thereby planar without any projections extending form the main plane of the second layer 1102b. In contrast the twelfth embodiment 1201 is arranged with tubes having a diameter of 3 mm and a height of 2 mm, wherein approximately 1 mm of the height of each tube extends out at either side of the structural arrangement 1202, creating a symmetrical semipermeable arrangement 1201.

The structural arrangement 1102, 1202 extends over the openings of the tubes 1103, 1203. However, the barrier provided by the coating 1181, 1281 of the tube 1103, 1203 prevents any lubricating fluid from tracking up the side of the tube 1103, 1203 and over the openings of the tube 1103, 1203. The openings thereby remain clear of any lubricating fluid when lubricating fluid is applied to the semipermeable arrangement 1101, 1201 and fluids such as water or air can pass freely through the semipermeable arrangement 1101, 1201. The portion of the structural means that extends over the tubes 1103, 1203 can be formed form chemically modified PTFE (e.g. with the addition of hydroxyl groups), heat-treated PTFE, or formed from an alternative polymeric, hydrophilic substance such as hydrophilic PU to ensure hydrophilicity and movement of water therethrough. The structural arrangement 1102, 1202 is further infused with perfluorocarbon liquid 20 to render the structural arrangement 1102, 1202 self-cleaning. The tubes 1103, 1203 contain soluble particles 70 of an antimicrobial substance but it should be noted that any desirable substance could be confined within the tubes 1103, 1203. For example, a medicament could be inserted were the semipermeable arrangement 1101, 1201 is to be used as a wound dressing or other clinical use, or activated charcoal could be inserted if the semipermeable arrangement 1101, 1201 is to be used as an air or water filter. The semipermeable arrangements 1101, 1201 can be used in a similar manner as described above.

Also provided by the invention is a method of manufacturing a semipermeable arrangement 1. Manufacturing the first embodiment involves forming a structural frame 2 from structural rods 4. The structural rods 4 are first formed by stretching PTFE into elongate rods. The structural rods 4 are then weaved together to form a single-layered mesh network 8 with a first group of structural rods 5 being spaced apart and parallel to one another and a second group of structural rods 6 being spaced apart and parallel to one another, the first group of structural rods 5 being arranged perpendicularly to the second group of structural rods 6. The mesh network 8 has openings 9 for receiving a porous member 3.

The porous members 3 are formed by sintering together loosely compacted particles of polypropylene to form roughly spherical porous members 3 with a diameter of 3 mm and pores sized less than or equal to 50 μm. The porous members 3 are then inserted into the openings 9 of the mesh network 8 as shown in FIG. 6. The porous members 3 are arranged by pressing them into the openings 8 such that they are retained by the structural frame 2. They are inserted to the extent that a portion of each porous member 8 extends above and below the plane of the structural frame 2 by around 0.5 to 1 mm. The method involves arranged the porous members 3 in rows leaving every other opening 9 empty. The adjacent rows have the same pattern but offset in relation to the rows either side such that the porous members 8 are in a diagonal relationship but are not longitudinally or laterally adjacent.

Next the method involves manufacturing a filter web 12 by electrospinning polyurethane to produce irregularly arranged filter web fibres 35 with gaps 36 therebetween defining pores as shown in FIGS. 17 to 22. The manufacturing process is controlled to produce pore sizes equal to or less than 1 μm after the filter web 12 is applied to the structural frame 2. The filter web 12 is then placed over the structural frame 2 with the porous members 3. An additional filter web 12 is then made in the same way and placed over the structural frame 2 and the porous members on the mutually opposing side of the semipermeable arrangement 1 creating a bilayer effect.

The embodiments having a self-cleaning web 514 that extends over the porous members 3, 103, 203, namely the fifth, ninth and tenth embodiments, are produced by manufacturing a self-cleaning web 514 by electrospinning PTFE to produce irregularly arranged self-cleaning web fibres 38 with gaps 39 therebetween defining pores as shown in FIGS. 23 to 25. The manufacturing process is controlled to ensure that the spacing between the self-cleaning web fibres 38 is suitable to permit capillary uptake of lubricant. The self-cleaning web 514 is then placed over the filter web 12 and the structural frame 2 with the porous members 3. As the self-cleaning web 514 is stretched over the porous members 3 the spacings between the self-cleaning web fibres 38 increases, reducing or eliminating the possibility of lubricant being dispersed through the self-cleaning web 514 at the porous members 3 by capillary action. An additional self-cleaning web 514 is then made in the same way and placed over the filter web 514, the structural frame 2 and the porous members 3 on the mutually opposing side of the semipermeable arrangement 1 creating a bilayer effect.

Manufacturing the second embodiment as illustrated in FIG. 7 involves the same steps as manufacturing the first embodiment with the additional steps of cutting holes in the self-cleaning web 14a, 14b corresponding to the location of the porous members 3 in the structural frame 2. When the self-cleaning web 14a, 14b is added to the structural frame 2 it does not cover the porous members 3. A further step involves exposing the semipermeable arrangement 201 to a solution of antimicrobial substance and allowing to dry to the form the soluble particles 70. Yet a further step involves adding perfluorocarbon liquid 20 to the self-cleaning web 14a.

Manufacturing the third embodiment as illustrated in FIG. 8 involves the same steps as manufacturing the second embodiment but replacing the spherical porous member 3 with a spherical cap porous member 103. Manufacturing the fourth embodiment (FIG. 9) is the same as manufacturing the second and third embodiments (FIGS. 7 and 8 respectively) but using a disc-shaped porous member 203. Manufacturing the fifth embodiment as illustrated in FIG. 10 is discussed above with the additional step of adding perfluorocarbon liquid 20 to the self-cleaning web 514a on one surface of the semipermeable arrangement 501. Manufacturing the sixth embodiment in FIG. 11 involves the same steps as manufacturing the second embodiment (FIG. 7) but with saturating the arrangement 601 with perfluorocarbon liquid such that it is present throughout the structural frame 2 and the self-cleaning webs 14a, 14b and an both surfaces of the semipermeable arrangement 601. Manufacturing the seventh embodiment as illustrated in FIG. 12 involves the same steps as manufacturing the sixth embodiment but replacing the spherical porous member 3 with a spherical cap porous member 103. Manufacturing the eighth embodiment (FIG. 13) is the same as manufacturing the sixth and seventh embodiments (FIGS. 11 and 12 respectively) but using a disc-shaped porous member 203. Manufacturing the ninth embodiment as illustrated in FIG. 14 involves the same steps as manufacturing the fifth embodiment (FIG. 10) however the perfluorocarbon liquid 20 is added throughout the structural frame 2 and on both self-cleaning webs 514a, 514b. Manufacturing the tenth embodiment as illustrated in FIG. 15 involves the same steps as manufacturing the ninth embodiment (FIG. 14) however the soluble antimicrobial particles 70 are not included.

The eleventh and twelfth embodiments are manufactured in a similar fashion. Initially, the tubes 1103, 1203 are prepared by forming an elongate tube of PTFE and heat shrinking an outer coating of FEP onto the PTFE tube. The tube has a diameter of 3 mm and is cut into a plurality of tubes 1103, 1203 of desired heights. Then the second layer 1102b, 1202b of ePTFE is set out and the tubes 1103, 1203 are arranged at desired locations on what will form part of the interior of the structural arrangement 1102, 1202. An ePTFE having a maximum pore size of 0.5 μm is selected as this is impermeable by pathogenic bacteria such as Staphylococcus aureus. Next, a soluble antimicrobial substance 70 is disposed in the tubes 1103, 1203. Then, the first layer 1102a, 1102b of ePTFE is applied over the second layer 1102b, 1202b and the tubes 1103, 1203 and is pressed onto the second layer 1102b, 1202b and the tubes 1103, 1203. The FEP of the tubes 1103, 1203 spreads into the pores of the structural arrangement (i.e. the gaps between fibrils of the ePTFE) and provides a barrier to movement of lubricating fluid. Finally, the structural arrangement 1102, 1202 is impregnated with perfluorocarbon liquid 20, rendering the structural arrangement 1102, 1202 self-cleaning.

An exemplary use of the semipermeable arrangement is illustrated in FIGS. 33 to 39 wherein two semipermeable arrangements of the eighth embodiment type 801a, 801b are arranged to form an envelope 50 formed for containing a MAVIED 52 and to be inserted into a recipient. The semipermeable arrangements 801a, 801b have a structural frame (not shown) and porous members (not shown) located within the structural frame. The envelope 50 is formed as a similar shape to a pulse generator, having a semicircular portion 53 and a rectangular portion 654 and a cavity 55 for receiving a pulse generator 56 as shown in FIGS. 36 to 38. The two semipermeable arrangements 801a, 801b are adjoined about their edges forming a side portion 57. The envelope 50 further has a seal arrangement 58 for sealing the envelope 50. The seal arrangement 58 extends along the rear edge portion of each of the semipermeable arrangements 51a, 51b and involves two mutually opposing seal members 59a, 59b—one situated on each semipermeable arrangement 801a, 801b—formed from ePTFE that can be pressed together to seal the envelope 50. One seal member 59a is a female sealing member with a groove extending along its length sized to receive male sealing member 59b (FIG. 39). The seal members 59a, 59b can be pressed together or pulled apart as required to seal and unseal the envelope 50 respectively.

The seal arrangement 58 further involves a reinforcement member 60 situated at one end of the two seal members 59a, 59b and holding them together. The reinforcement member 60 provides additional strength to the structure of the envelope 50 and prevents the two semipermeable arrangements 801a, 801b from easily being torn apart. The envelope 50 further has an exit point 61 located at the side portion 57 at the end of the two sealing members 59a, 59b to permit passage of a lead 62 from inside the cavity 55 to the outside of the envelope 50. The envelope 50 further has an extended portion 63 that extends out from the cavity 55 and is adapted for extending along a length of a lead 62.

In use, as shown in FIGS. 36 to 38, a pulse generator 56 is inserted into the cavity 55 of the envelope 50 and the redundant lead length of the pulse generator 56 is also folded into the cavity 55 on the top or the bottom of the pulse generator 56. The lead 62 is arranged extending along the extended portion 63 of the envelope 50 and out the exit point 61. The envelope 50 is sealed by operating the seal arrangement 58. Specifically, the two seal members 59a, 59b are pressed to close the seal arrangement 58. Finally, a suture 65 is tied around the extended portion 63 thereby sealing the envelope. The envelope 50 can then be inserted into a recipient. The semipermeability of the semipermeable arrangements 801a, 801b allows water and dissolved substances to enter the envelope but prevents entry of bacteria. The pulse generator can therefore provide electrical current across the envelope. The self-cleaning arrangement of the semipermeable arrangements 801a, 801b prevents adhesion of bacteria or formation of tissue on the surface of the envelope 50.

In the preceding discussion of the invention, unless stated to the contrary, the disclosure of alternative values for the upper or lower limit of the permitted range of a parameter, coupled with an indication that one of the values is more highly preferred than the other, is to be construed as an implied statement that each intermediate value of the parameter, lying between the more preferred and the less preferred of the alternatives, is itself preferred to the less preferred value and also to each value lying between the less preferred value and the intermediate value.

The features disclosed in the foregoing description or the following drawings, expressed in their specific forms or in terms of a means for performing a disclosed function, or a method or a process of attaining the disclosed result, as appropriate, may separately, or in any combination of such features be utilised for realising the invention in diverse forms thereof as defined in the appended claims.

1.-39. (canceled)

40. A semipermeable arrangement for use in clinical, agricultural, industrial and/or environmental settings comprising a porous structural means formed from one or more layers, the structural means being formed at least partially from a material having an affinity to a lubricating fluid, the lubricating fluid being perfluorocarbon liquid, and being adapted such that it can be infused with a lubricating fluid, the semipermeable arrangement being arranged with means to prevent or limit the movement of the lubricating fluid through at least part of the structural means such that when the lubricating fluid is applied to the structural means, the movement of lubricating fluid through the structural means is restricted, and there are areas on the semipermeable arrangement that are completely or substantially free from the presence of lubricating fluid, the means to prevent or limit movement of the lubricating fluid through at least part of the structural means further being arranged having one or more passageways to permit the movement of fluids such as air, water and dissolved substances through the structural means.

41. The semipermeable arrangement as claimed in claim 40, wherein the structural means comprises PTFE.

42. The semipermeable arrangement as claimed in claim 41, wherein the means to prevent or limit movement of the lubricating fluid through at least part of the structural means includes barriers located within the pores of the structural means.

43. The semipermeable arrangement as claimed in claim 42, wherein the barriers are provided by pressing and/or heat-melting a polymer such as PU or FEP into the pores of the structural means.

44. The semipermeable arrangement as claimed in claim 43, wherein the means to prevent or limit movement of the lubricating fluid through at least part of the structural means is retained within the structural means.

45. The semipermeable arrangement as claimed in claim 44, wherein the means to prevent or limit movement of the lubricating fluid through at least part of the structural means extends out from a main plane of the structural means in at least one direction.

46. The semipermeable arrangement as claimed in claim 45, wherein the means to prevent or limit movement of the lubricating fluid through at least part of the structural means is disposed between first and second layers of the structural means.

47. The semipermeable arrangement as claimed in claim 46, wherein the means to prevent or limit the movement of the lubricating fluid through at least part of the structural means comprises at least one substantially hollow member, the substantially hollow member having a first opening arranged at one side of the structural means and a second opening arranged at another side of the structural means.

48. The semipermeable arrangement as claimed in claim 47, wherein the substantially hollow member is formed at least partially from one or more polymeric substances, for example polyurethane (PU), fluorinated ethylene propylene (FEP) and/or PTFE.

49. The semipermeable arrangement as claimed in claim 48, wherein the substantially hollow member is a tube.

50. The semipermeable arrangement as claimed in claim 49, wherein the structural means comprises a plurality of fibrils and wherein the spacing between the fibrils is equal to or less than 1.0 μm.

51. The semipermeable arrangement as claimed in claim 47, wherein the means to prevent or limit movement of the lubricating fluid through at least part of the structural means comprises at least one porous member.

52. The semipermeable arrangement as claimed in claim 51, wherein the porous member is spherical, roughly spherical, or a flattened sphere, spherical cap, hemisphere, ovoid, cube or cuboid in shape.

53. The semipermeable arrangement as claimed in claim 40, comprising lubricating fluid.

54. The semipermeable arrangement as claimed in claim 53, comprising perfluorocarbon liquid.

55. The semipermeable arrangement as claimed in claim 40, wherein the passageways of the means to prevent or limit movement of the lubricating fluid through at least part of the structural means comprises additives.

56. The semipermeable arrangement as claimed in claim 55, wherein the additives comprise soluble antimicrobial substances or medicaments, and/or a water and/or air-purifying substance.
